SEQUENCE LISTING (28 sequences)

SEQ ID NO: 1 - protein, 459 aa, Triticum aestivum
MDIDHGRESDGEMVGTIASCGLLLHSLLAGLGRRAAGFARKVGGAAREDPRRVAHSLKVGLALALVSVVYFVTPLFNGLGVSAIWAVLTVVVVMEYTVGATLSKGLNRALATLVAGCIAVGAHQLAELAERCGDQGEPIVLTVLVFFVASAATFLRFIPEIKAKYDYGVTIFILTFGLVAVSSYRVEELIQLAHQRFYTIAVGVFICLCTTVFLFPVWAGEDVHKLASGNLDKLAQFIEGMEFNCFGENSVANNFGGKDSPQMHKSVLNSKATEDSLCTFAKWEPRHGQFRFRHPWSQYQKLGTLCRQCASSMEALASYVITTSKTQCPAAANPELSCKVRKTCGEMSLHSSKVLRDLAMATRTMTVPSPVNITMATAVKAAESLRSELAENTALLQVMHVAVTATLLADLVDRVKEIAECVDVLARLAHFKNPEDTKNVVVSTVSRGIDEPLPDVVIL

SEQ ID NO: 2 - DNA, 1380 bp, Triticum aestivum
atggatattgatcacggcagagagagcgacggcgagatggtgggcaccatcgccagctgcgggctgctgctccactcgcttctcgccgggctcgggcgtcgcgccgccgggttcgcccggaaggtgggcggcgccgcgcgggaggacccgaggcgggtggcgcactcgctcaaagtcggcctggcgctcgcgctggtgtccgtcgtctacttcgtcacgccgctcttcaacggcctcggggtgtccgcgatatgggccgtgctcaccgtcgtcgtcgtcatggagtacaccgtcggtgccacgctgagtaaaggcttgaacagagccttggcgacgttggtggctggctgcatcgccgtcggagctcatcagttagctgaattagctgaacgctgtggtgatcagggagagcccatagtgctcaccgtgctcgtcttcttcgtagcgtcagcggcgacgttcttgcgcttcatcccggagatcaaggccaagtacgactacggcgtgaccatcttcatactgaccttcggtctggtggccgtgtcgagctacagagtggaggagctcatccagctcgcgcaccagcggttctacaccatagccgtcggcgtcttcatctgcctctgcaccaccgtcttcctcttccccgtctgggccggagaggacgtccacaagctcgcctccggcaacctcgacaaactcgctcagttcattgaaggaatggaattcaactgctttggcgaaaacagtgttgcaaataattttgggggaaaggattccccccaaatgcacaagagcgtccttaattcgaaggccactgaggactctttgtgcacctttgccaaatgggagcctcgtcatggccagttcagatttcgacacccatggagccaataccagaagctgggaactctttgtcgccaatgtgcgtcttctatggaggctcttgcttcatatgtcatcacaacctcaaaaacccagtgccctgctgcagccaaccctgagctatcatgtaaggttcgaaaaacatgtggcgaaatgagcttgcattcctccaaggtgcttagggatctcgcaatggcaactcgaacaatgactgtgccgtctccagtgaatatcaccatggctacagccgtgaaagcagcggaaagcctcagaagcgagcttgcagagaacacggctctgttgcaagtgatgcatgtggccgtcaccgcaacacttcttgcggacttggttgatagggtgaaggaaatcgcggaatgtgttgatgtcctagcaagactggcgcactttaagaaccccgaggacacaaaaaatgtcgttgtcagtaccgtgagtcgagggatagacgaacctttgcctgacgtggttattttgtaa

SEQ ID NO: 3 - protein, 493 aa, Arabidopsis thaliana
MEKVREIVREGIRVGNEDPRRIIHAFKVGLALVLVSSFYYYQPFGPFTDYFGINAMWAVMTVVVVFEFSVGATLGKGLNRGVATLVAGGLGIGAHQLARLSGATVEPILLVMLVFVQAALSTFVRFFPWVKTKFDYGILIFILTFALISLSGFRDEEIMDLAESRLSTVVIGGVSCILISIFVCPVWAGQDLHSLLASNFDTLSHFLQDFGDEYFEAREKGDYKVVEKRKKNLERYKSVLDSKSDEEALANYAEWEPPHGQFRFRHPWKQYVAVGALLRQCAYRIDALNSYINSDFQIPVDIKKKLETPLRRMSSESGNSMKEMSISLKQMIKSSSSDIHVSNSQAACKSLSTLLKSGILNDVEPLQMISLMTTVSMLIDIVNLTEKISESVHELASAARFKNKMRPTVLYEKSDSGSIGRAMPIDSHEDHHVVTVLHDVDNDRSNNVDDSRGGSSQDSCHHVAIKIVDDNSNHEKHEDGEIHVHTLSNGHLQ

SEQ ID NO: 4 - DNA, 1482 bp, Arabiposis thaliana
atggagaaagtgagagagatagtgagagaagggattagagtagggaatgaagacccgagaagaattattcatgctttcaaagtgggacttgctcttgttttggtctcttccttctattattaccaacctttcggtcctttcaccgattacttcggtatcaatgcaatgtgggctgtcatgaccgtcgttgttgtctttgaattctccgtaggggctacacttgggaaaggactaaatagaggagtggcaacattagtagcaggaggactaggaattggagctcatcaactagcaagattgtcgggtgcaacagtggaacctattcttttggtcatgttggtctttgtgcaagctgcgttgtcgacgttcgtaaggtttttcccatgggtaaagacaaagttcgattatgggatattgatattcatattgacgttcgcgctgatatcgttgtcggggtttagagacgaagagataatggatttggcagaaagcagactatcgacagtggtgattggaggagtcagttgcatccttatctctatctttgtctgtcctgtttgggctggacaagatctacattctctccttgcctctaactttgacactctctcccacttccttcaagattttggggacgaatattttgaagcgagagagaagggggactacaaagtagtggagaagaggaaaaagaatcttgagagatataaaagtgttcttgactccaaaagcgatgaagaagctttggctaattacgcagaatgggaaccgcctcatggacaatttaggtttaggcatccatggaaacaatatgttgccgttggtgcattactccggcagtgtgcctacaggattgatgccttgaattcatacatcaactcagattttcagatcccagtggacataaaaaagaaattagaaacaccattaagaagaatgagctcggaatcgggaaactcaatgaaagaaatgtccatttcactaaaacaaatgataaaatcatcttcttctgatatccatgtctcaaactcacaagctgcatgcaaatctctttctactttacttaaatcaggcatcttgaacgatgttgagcctcttcaaatgatctcattgatgacgactgtctccatgctcatagatattgttaacttaaccgaaaagatctcggaatctgtacacgagcttgcatccgctgcaagatttaagaacaagatgaggccgaccgtgctatacgagaagtcggattccggaagcattggtcgtgccatgccaatagattctcatgaagatcatcatgttgtcacagttttacatgacgttgataatgatagatcaaacaatgttgatgattcacgtggagggagttcacaggactcatgtcatcatgttgccataaagattgttgatgataactcaaaccatgaaaaacatgaagatggtgaaatacatgtacatacattaagtaatgggcatcttcagtaa

SEQ ID NO: 5 - DNA, 621 bp, Hordeum vulgare
cggcacgaggcgaggatctccacaagctcaccgccgccaacctcgataagctggcgcagtttcttcaaggattggaatccgaatgttttggagagaaggctgctagcgagaatttggaggacaaagcctttctgcaagtgtacaagagcgtcctcaactccaaggccagtgaggactctctgagcaattttgccaagtgggagcccggtcatggcaaattcggcttccggcacccatggagccaataccagaagctcggagctctttgtcgtcagtgcgcatcttcaatggaggctcttgcttcctatgtcatcacactgcaaaaatcccagtaccctgaggccaatccagagctaaccttcaaggtccgaatggcgtgtggggaaatgagctcacactcggccaaggcgctcaaggacctatcaacagccattcggacaatgatagtaccatctccagccaacatcaccatgtcctcagccatcaaagttgcaaaagacctcaggaatgaattatcagaggacgcagctgtgttgcaagtgatgcatgtggctgttactgccacacttatctcagacttggttacgacaatagtgaaaattgcagaaactgctgacaacc

SEQ ID NO: 6 - DNA, 1368 bp, Oryza sativa
atgcaatctgctgccgtggagacgacgccggcggcggcggcggcggcggagaacggcggccaagtggtcgtcgtcgtcggcggggggtgggtggatggatgctgggcgcgggtgcgagcggcggtggaggtggccgggaggtgggtgggcgggctggcgaggaaggtgggcgggatcgcggcggacgacccgaggagggtggcgcactcgctcaaggtcgggctggcgctgacgctggtgtccgtgctctactacgtgacgccgcttttcaaggggttcggggtctccacgctctgggccgtgctcaccgtcgtcgtcgtcatggagtacaccgtcggtggcacgctgagcaaagggttgaacagggccttcgcgacgctggtggctgggttcatcgccgtcggggcgcatcaggtggctaaccggtgcggcgcacagggagagcccatactcctcgccgtcttcgtcttcctactagcgtcggcggcgacgttctcgcggttcataccggagataaaggcgaggtacgactacggggtgaccatcttcatcctgaccttcagcctggtggccgtgtcgagctaccgggtagaggagctcatccagctcgcgcaccagcgcttctccaccatcgtcgtcggcgtcgccacctgcctctgcaccaccatcttcgtcatgcccgtctgggccggcgaggacctccacaagctcgccgccggcaacctcgacaagctggccgacttccttgaaggaatggaaaccgaatgctttggagaaagcgctacgagtgagagtttggagggcaaagcctttctccaagcgtacaagagcatacttaattcgaaggccactgaagactctctgtgcaactttgccagatgggagcctggtcatggaaaattcagtttcaaacacccatggagccaataccagaagattggagctctttctcgccagtgcgcttcttccatggaggctatggcttcctatgtcatcacactcacaaaatctcagtatcctgaagccaatcctgagctgtccttcaaagtacgaacagcatgcagtgagatgagctcacactctgctcaggcactcagagagctctcggcagcacttcgaacaatgaccgtaccatctacaacctccatgtctgcagccatcaaagctgccaaaacactcaggagcgagctatcagaggataaagctctgctgcaagtgatgcatgtggcagttactgcatcacttctttcagacttggttacacaagtaaagaaaattgcagaatctgtcgataacctggcacgacttgcctgcttcaaagtccctgaaaaatctcagaaagaagtggctatcaacatcatgagttga

SEQ ID NO: 7 - DNA, 1559 bp, Arabidposis thaliana
catcttttctttctatctgaaagtaatcagagaatcagaaaacacttgagagagctgagtgaccatcaaaaggtgttatggagaaagtgagagagatagtgagagaagggattagagtagggaatgaagacccgagaagaattattcatgctttcaaagtgggacttgctcttgttttggtctcttccttctattattaccaacctttcggtcctttcaccgattacttcggtatcaatgcaatgtgggctgtcatgaccgtcgttgttgtctttgaattctccgtaggggctacacttgggaaaggactaaatagaggagtggcaacattagtagcaggaggactaggaattggagctcatcaactagcaagattgtcgggtgcaacagtggaacctattcttttggtcatgttggtctttgtgcaagctgcgttgtcgacgttcgtaaggtttttcccatgggtaaagacaaagttcgattatgggatattgatattcatattgacgttcgcgctgatatcgttgtcggggtttagagacgaagagataatggatttggcagaaagcagactatcgacagtggtgattggaggagtcagttgcatccttatctctatctttgtctgtcctgtttgggctggacaagatctacattctctccttgcctctaactttgacactctctcccacttccttcaagattttggggacgaatattttgaagcgagagagaagggggactacaaagtagtggagaagaggaaaaagaatcttgagagatataaaagtgttcttgactccaaaagcgatgaagaagctttggctaattacgcagaatgggaaccgcctcatggacaatttaggtttaggcatccatggaaacaatatgttgccgttggtgcattactccggcagtgtgcctacaggattgatgccttgaattcatacatcaactcagattttcagatcccagtggacataaaaaagaaattagaaacaccattaagaagaatgagctcggaatcgggaaactcaatgaaagaaatgtccatttcactaaaacaaatgataaaatcatcttcttctgatatccatgtctcaaactcacaagctgcatgcaaatctctttctactttacttaaatcaggcatcttgaacgatgttgagcctcttcaaatgatctcattgatgacgactgtctccatgctcatagatattgttaacttaaccgaaaagatctcggaatctgtacacgagcttgcatccgctgcaagatttaagaacaagatgaggccgaccgtgctatacgagaagtcggattccggaagcattggtcgtgccatgccaatagattctcatgaagatcatcatgttgtcacagttttacatgacgttgataatgatagatcaaacaatgttgatgattcacgtggagggagttcacaggactcatgtcatcatgttgccataaagattgttgatgataactcaaaccatgaaaaacatgaagatggtgaaatacatgtacatacattaagtaatgggcatcttcagtaa

SEQ ID NO: 8 - DNA, 20 bp, Artificial sequence
  Oligonucleotide primer
cgtgaaagcagcggaaagcc

SEQ ID NO: 9 - DNA, 25 bp, Artificial sequence
  Oligonucleotide primer
ccctcgactcacggtactaacaacg

SEQ ID NO: 10 - DNA, 20 bp, Artificial sequence
  Oligonucleotide primer
aacaagactgctttcaccac

SEQ ID NO: 11 - DNA, 20 bp, Artificial sequence
  Oligonucleotide primer
tctcagaaagctcacggtag

SEQ ID NO: 12 - DNA, 20 bp, Artificial sequence
  Oligonucleotide primer
tctcagagagctcacggtag

SEQ ID NO: 13 - DNA, 22 bp, Artificial sequence
  Oligonucleotide primer
gaaggacatcttcacggcgatc

SEQ ID NO: 14 - DNA, 20 bp, Artificial sequence
  Oligonucleotide primer
cacggccatgaagaagaagc

SEQ ID NO: 15 - DNA, 20 bp, Artificial sequence
  Oligonucleotide primer
caccgccatgaagaagaatc

SEQ ID NO: 16 - DNA, 19 bp, Artificial sequence
  Oligonucleotide primer
ctgatcttctgtgaagggt

SEQ ID NO: 17 - DNA, 20 bp, Artificial sequence
  Oligonucleotide primer
tgatagaactcgtaatgggc

SEQ ID NO: 18 - DNA, 22 bp, Artificial sequence
  Oligonucleotide primer
gcgtggaccgcttgctgcaact

SEQ ID NO: 19 - DNA, 25 bp, Artificial sequence
  Oligonucleotide primer
gaagctgcaagaaaaagtttagtta

SEQ ID NO: 20 - DNA, 21 bp, Artificial sequence
  Oligonucleotide primer
ccactgttgcacccgacaatc

SEQ ID NO: 21 - DNA, 22 bp, Artificial sequence
  Oligonucleotide primer
atggagaaagtgagagagatag

SEQ ID NO: 22 - DNA, 2271 bp, Hordeum vulgare cv Morex
atggaggttgatcaccgcatcagggtgagcgacggcgacggcgagacgacggccggacaaggaggcgttgttgccggcgtctcgttcgcgggctgctggcagcggctccggtcggtgctcgtggggctgtggtgttgggtcgccgtgttcgcgaggaaggtgggcaggatcgcgagggaggacccacggcgggtggcgcactcgctcaaggtcggcctggcgctcaccctggtgtccgtcctctactacgtcacgccgctcttcaagggcttcggggtctccacgatgtgggccgtgctcaccgtcgtcgtcgtcatggagtacaccgtcggtccgtgtcgtcatacttgttacagccgcaccatattagtattagtatacgtatatgtgtatgtgtgtctctgaacctgaacgtgcgctgcatgcatgcatgcaacaggtggcacgctgagcaaaggcttgaatagagctttcgcgacgctggtggctgggttcatcgccgtgggagctcatcaggtagctaaccgctgtggtgcacaaggggagcccatactgctcgccatcttcgtcttcttcctaggtatactatactactacgttttcggatgcatacgcatggtccggcaactactcctttcattcctaaaaatacaagtcttttttagagattttattatcaactacatacaaaacaaaatagatgaatcaacattctaagatttgtctatatacatctatatgtagcctctaattgaatctctaaagagacttatatttagaaacggattatatatatatgtattgcagcgtcggcggcgacgttctcgcgcttcatcccggagatcaaggcgaggtacgactacggcgtgaccatcttcatactcaccttcagcctggtggccgtgtccagctaccgcgtggaggagctcattcagctggcgcaccagcgcttctccaccatcgtcatcggcgtccttacctgcctctgcaccaccatcttcgtcttccccgtctgggccggcgaggatctccacaagctcaccgccgccaacctcgataagctggcgcagtttcttcaaggtaatcactatactttttctattcctgcccctttttgtcactctcgttcaagttttcaatggtatacgaatgcaacttgatttgtgttctttggaaagaaaaataaagaaaggaacatcaaacggtggagttttcctgacaagagttcaccagacatactctgttttatactagttctgcttctcccctgtcgaacctctttagcaacttcatatcggtacaaaagagtttgtgagactgaaaactgtcaccagacatactctgttttatactagttctgcttctcccctgtcgaacctctgtagcaacttcatatcggtacaaaagagtttgtgagactgaaaactgagcacatcgtacgcaggattggaatccgaatgttttggagagaaggctgctagcgagaatttggaggacaaagcctttctgcaagtgtacaagagcgtcctcaactccaaggccagtgaggactctctggtaagcttcctcacagactattttgacggtgaactatatgctcagcgcagaagctgcaataaacgtctgcaacaagacagcttgtacacacggcagtttttgtaatgttattctgaaaattgcagagcaattttgccaagtgggagcccggtcatggcaaattcggcttccggcacccatggagccaataccagaagctcggagctctttgtcgtcagtgcgcatcttcaatggaggctcttgcttcctatgtcatcacactgcaaaaatcccaggtaattgcagatcctcccccactccatgatcttatacggagactactacgggcccaacaacatttttcctcatcaacagtaccctgaggccaatccagagctaaccttcaaggtccgaatggcgtgtggggaaatgagctcacactcggccaaggcgctcaaggacctatcaacagccattcggacaatgatagtaccatctccagccaacatcaccatgtcctcagccatcaaagttgcaaaagacctcaggaatgaattatcagaggacgcagctgtgttgcaagtgatgcatgtggctgttactgccacacttatctcagacttggttacgacaatagtgaaaattgcagaaactactgacaacctagcacggcttggccacttcaagaaccctgaaaaaactcagaaagatgtcgctatcaacattgcgagttga

SEQ ID NO: 23 - DNA, 1368 bp, Hordeum vulgare cv Dayton
atggaggttgatcaccgcatcagggtgagcgacggcgacggcgagacgacggccggacaaggaggcgttgttgccggcgtctcgttcgcgggctgctggcagcggctccggtcggtgctcgtggggctgtggtgttgggtcgccgtgttcgcgaggaaggtgggcaggatcgcgagggaggaccctcggcgggtggcgcactcgctcaaggtcggcctggcgctcaccctggtgtccgtcctctactacgtcacgccgctcttcaagggcttcggggtctccacgatgtgggccgtgctcaccgtcgtcgtcgtcatggagtacaccgtcggtggcacgctgagcaaaggcttgaatagagctttcgcgacgctggtggctgggttcatcgccgtgggagctcatcaggtagctaaccgctgtggtgcacaaggggagcccatactgctcgccatcttcgtcttcttcctagcgtcggcggcgacgttctcgcgcttcatcccggagatcaaggcgaggtacgactacggcgtgaccatcttcatactcaccttcagcctggtggccgtgtccagctaccgcgtggaggagctcattcagctggcgcaccagcgcttctccaccatcgtcatcggcgtccttacctgcctctgcaccaccatcttcgtcttccccgtctgggccggcgaggatctccacaagctcaccgccgccaacctcgataagctggcgcagtttcttcaaggattggaatccgaatgttttggagagaaggctgctagcgagaatttggaggacaaagcctttctgcaagtgtacaagagcgtcctcaactccaaggccagtgaggactctctgagcaattttgccaagtgggagcccggtcatggcaaattcggcttccggcacccatggagccaataccagaagctcggagctctttgtcgtcagtgcgcatcttcaatggaggctcttgcttcctatgtcatcacactgcaaaaatcccagtaccctgaggccaatccagagctaaccttcaaggtccgaatggcgtgtggggaaatgagctcacactcggccaaggcgctcaaggacctatcaacagccattcggacaatgatagtaccatctccagccaacatcaccatgtcctcagccatcaaagttgcaaaagacctcaggaatgaattatcagaggacgcagctgtgttgcaagtgatgcatgtggctgttactgccacacttatctcagacttggttacgacaatagtgaaaattgcagaaactgctgacaacctagcacggcttggccacttcaagaaccctgaaaaaactcagaaagatgtcgctatcaacattgcgagttga

SEQ ID NO: 24 - protein, 455 aa, Hordeum vulgare cv Dayton
MEVDHRIRVSDGDGETTAGQGGVVAGVSFAGCWQRLRSVLVGLWCWVAVFARKVGRIAREDPRRVAHSLKVGLALTLVSVLYYVTPLFKGFGVSTMWAVLTVVVVMEYTVGGTLSKGLNRAFATLVAGFIAVGAHQVANRCGAQGEPILLAIFVFFLASAATFSRFIPEIKARYDYGVTIFILTFSLVAVSSYRVEELIQLAHQRFSTIVIGVLTCLCTTIFVFPVWAGEDLHKLTAANLDKLAQFLQGLESECFGEKAASENLEDKAFLQVYKSVLNSKASEDSLSNFAKWEPGHGKFGFRHPWSQYQKLGALCRQCASSMEALASYVITLQKSQYPEANPELTFKVRMACGEMSSHSAKALKDLSTAIRTMIVPSPANITMSSAIKVAKDLRNELSEDAAVLQVMHVAVTATLISDLVTTIVKIAETADNLARLGHFKNPEKTQKDVAINIAS

SEQ ID NO: 25 - DNA, 1368 bp, Hordeum vulgare cv Morex
atggaggttgatcaccgcatcagggtgagcgacggcgacggcgagacgacggccggacaaggaggcgttgttgccggcgtctcgttcgcgggctgctggcagcggctccggtcggtgctcgtggggctgtggtgttgggtcgccgtgttcgcgaggaaggtgggcaggatcgcgagggaggacccacggcgggtggcgcactcgctcaaggtcggcctggcgctcaccctggtgtccgtcctctactacgtcacgccgctcttcaagggcttcggggtctccacgatgtgggccgtgctcaccgtcgtcgtcgtcatggagtacaccgtcggtggcacgctgagcaaaggcttgaatagagctttcgcgacgctggtggctgggttcatcgccgtgggagctcatcaggtagctaaccgctgtggtgcacaaggggagcccatactgctcgccatcttcgtcttcttcctagcgtcggcggcgacgttctcgcgcttcatcccggagatcaaggcgaggtacgactacggcgtgaccatcttcatactcaccttcagcctggtggccgtgtccagctaccgcgtggaggagctcattcagctggcgcaccagcgcttctccaccatcgtcatcggcgtccttacctgcctctgcaccaccatcttcgtcttccccgtctgggccggcgaggatctccacaagctcaccgccgccaacctcgataagctggcgcagtttcttcaaggattggaatccgaatgttttggagagaaggctgctagcgagaatttggaggacaaagcctttctgcaagtgtacaagagcgtcctcaactccaaggccagtgaggactctctgagcaattttgccaagtgggagcccggtcatggcaaattcggcttccggcacccatggagccaataccagaagctcggagctctttgtcgtcagtgcgcatcttcaatggaggctcttgcttcctatgtcatcacactgcaaaaatcccagtaccctgaggccaatccagagctaaccttcaaggtccgaatggcgtgtggggaaatgagctcacactcggccaaggcgctcaaggacctatcaacagccattcggacaatgatagtaccatctccagccaacatcaccatgtcctcagccatcaaagttgcaaaagacctcaggaatgaattatcagaggacgcagctgtgttgcaagtgatgcatgtggctgttactgccacacttatctcagacttggttacgacaatagtgaaaattgcagaaactactgacaacctagcacggcttggccacttcaagaaccctgaaaaaactcagaaagatgtcgctatcaacattgcgagttga

SEQ ID NO: 26 - protein, 455 aa, Hordeum vulgare cv Morex
MEVDHRIRVSDGDGETTAGQGGVVAGVSFAGCWQRLRSVLVGLWCWVAVFARKVGRIAREDPRRVAHSLKVGLALTLVSVLYYVTPLFKGFGVSTMWAVLTVVVVMEYTVGGTLSKGLNRAFATLVAGFIAVGAHQVANRCGAQGEPILLAIFVFFLASAATFSRFIPEIKARYDYGVTIFILTFSLVAVSSYRVEELIQLAHQRFSTIVIGVLTCLCTTIFVFPVWAGEDLHKLTAANLDKLAQFLQGLESECFGEKAASENLEDKAFLQVYKSVLNSKASEDSLSNFAKWEPGHGKFGFRHPWSQYQKLGALCRQCASSMEALASYVITLQKSQYPEANPELTFKVRMACGEMSSHSAKALKDLSTAIRTMIVPSPANITMSSAIKVAKDLRNELSEDAAVLQVMHVAVTATLISDLVTTIVKIAETTDNLARLGHFKNPEKTQKDVAINIAS

SEQ ID NO: 27 - DNA, 21 bp, Artificial Sequence
  Oligonucleotide primer
atggaggttgatcaccgcatc

SEQ ID NO: 28 - DNA, 22 bp, Artificial Sequence
  Oligonucleotide primer
tcaactcgcaatgttgatagcg

(19) United States
(12) Patent Application Publication
(10) Pub. No.: US 2012/0185976 A1
(43) Pub. Date: Jul. 19, 2012

(54) ALUMINIUM TOLERANT BARLEY

(75) Inventors: Peter Richard Ryan, Watson (AU); Emmanuel Delhaize, Giralang (AU)

(73) Assignee: COMMONWEALTH SCIENTIFIC AND INDUSTRIAL RESEARCH ORGANISATION

(21) Appl. No.: 13/305,383

(22) Filed: Nov. 28, 2011

Related U.S. Application Data
(63) Continuation of application No. 11/631,698, filed on Jun. 29, 2007, now abandoned.

(30) Foreign Application Priority Data
Jul. 7, 2004 (AU) 2004903739

(51) Int. Cl. A01H 5/00; A01H 5/10; C08B 30/00

(52) U.S. Cl. 800/320; 536/102

(57) ABSTRACT

The present invention relates to barley plants comprising an exogenous nucleic acid molecule which confers upon the plants enhanced tolerance to aluminium relative to isogenic plants which do not contain the exogenous nucleic acid. Also provided are methods of producing barley plants with enhanced tolerance to aluminium.

### FIELD OF THE INVENTION

The present invention relates to barley plants comprising an exogenous nucleic acid molecule which confers upon the plants enhanced tolerance to aluminium relative to isogenic plants which do not contain the exogenous nucleic acid molecule. Also provided are methods of producing barley plants with enhanced tolerance to aluminium.

### BACKGROUND OF THE INVENTION

Acid soils cover some 40% of the Earth's arable land and represent a major limitation to plant production (von Uexküll and Mutert, 1995). The main constraint to plant growth on these soils is the presence of toxic aluminium cations (Al³⁺) that are solubilized by the acidity and which rapidly inhibit root. Aluminium (Al) toxicity is manifested primarily by the inhibition of root growth which results in limited uptake of water and nutrients (Kochian, 1995). Plant production on acid soils can be maintained by neutralizing the acidity with lime (CaCO₃) and through the use of aluminium tolerant plant species. However, lime is ineffective in correcting acidity at depth and many important crop and pasture species lack sufficient Al tolerance within their germplasm to allow effective breeding for this character.

Genetic engineering provides an opportunity to enhance the Al tolerance of sensitive species through the over-expression of endogenous genes or by the expression of foreign genes. Towards this end, the Al tolerance of canola (Brassica napus), Arabidopsis thaliana, tobacco (Nicotiana tabacum) and alfalfa (Medicago sativum) have been reported to be enhanced by increasing organic acid biosynthesis through over-expression of citrate synthase or malate dehydrogenase genes derived from plants or bacteria (Anoop et al., 2003; Koyama et al., 2000; de la Fuente et al., 1997; Tesfaye et al., 2001). In some cases this has lead to increased secretion of organic acids from roots and the increased Al tolerance was attributed to the ability of organic acids to chelate and detoxify Al³⁺. However, the increases in Al tolerance have at best been modest or, as in the case of a Psuedomonas aeuriginosa citrate synthase gene, not easily reproducible (Delhaize et al., 2000). While these plants might have increased biosynthesis of organic acids, the transport of these molecules to the external medium appears to be a rate limiting step (Ryan et al., 2001).

A gene (ALMT1) from wheat (Triticum aestivum) encoding a protein with properties consistent with it being an Al-activated channel for malate efflux has recently been cloned (Sasaki et al., 2004). The ALMT1 protein is membrane bound, the gene co-segregates with Al tolerance in populations of wheat plants, and expression of ALMT1 in Xenopus oocytes, rice (Oryza sativa) and tobacco cells conferred an Al-activated efflux of malate.

Barley (Hordeum vulgare) is an economically important crop in many parts of the world and is among the most Al-sensitive of the cereal crops (Zhao et al., 2003). There is relatively little variation for aluminium tolerance in barley, and hence there is a need for Al-tolerant varieties.

### SUMMARY OF THE INVENTION

The present inventors have observed that whilst an ALMT1 gene expressed in rice or tobacco cells is able to confer upon these cells the ability to efflux malate upon exposure to aluminium, this gene did not result in rice or tobacco plants with enhanced tolerance to aluminum. These experimental observations suggest that aluminium tolerance may not necessarily be conferred upon an aluminium sensitive non-wheat plant by the expression of a single transgene, particularly a single transgene encoding an aluminium activated organic acid transporter such as ALMT1. However, the present inventors surprisingly found that despite the inability to produce transgenic rice or tobacco with enhanced aluminium tolerance they were able to produce barley with this trait. As a result, the present inventors are the first to produce barley with enhanced tolerance to aluminium when compared to wild-type plants.

Thus, in a first aspect, the present invention provides a barley plant comprising an exogenous nucleic acid molecule, wherein the barley plant has enhanced tolerance to aluminium relative to an isogenic plant not having the exogenous nucleic acid molecule.

The plants of the first aspect may be produced by methods including the use of transgenic techniques, as well as plant breeding procedures such as, for example, introgression of exogenous genes, to produce the barley plant. In one embodiment, the barley plant is transgenic. In another embodiment, the barley plant has one or more introgressed genes from a source other than barley. In a particular embodiment, the introgressed gene is an ALMT1 gene from wheat or a progenitor of wheat.

In one embodiment, the barley plant, when grown hydroponically in a medium consisting of an hydroponic growth solution having a defined concentration of aluminium chloride, grows without significant inhibition of root growth relative to growth when grown hydroponically in a medium consisting of said hydroponic growth solution without aluminium chloride, wherein the hydroponic growth solution consists of distilled or deionized water with added salts at the concentrations: 500 μM KNO₃, 500 μM CaCl₂, 500 μM NH₄NO₃, 150 μM MgSO₄, 10 μM KH₂PO₄, 2 μM FeCl₃, 11 μM H₃BO₃, 2 μM MnCl₂, 0.35 μM ZnCl₂ and 0.2 μM CuCl₂ and which is adjusted to pH 4.3, and wherein the defined concentration of aluminium chloride is at least about 5 μM.

The plants can be grown for any length of time sufficient to determine whether aluminium is inhibiting root growth. In one embodiment, the plants are grown for at least 7 days, preferably at least 14 days.

In further embodiments, the defined concentration of aluminium is at least about 10 μM or at least about 20 μM.

In another embodiment, the barley plant comprises a transgene encoding a polypeptide having aluminium activated organic acid transporter activity. Without being limited by this model, the organic acid transported by the polypeptide can be any organic acid known, or found to be, secreted by plants which has a capability to chelate aluminium in soils. The organic acid may be a di-carboxylic acid or a tri-carboxylic acid. The organic acid transporter may lead to the efflux of one or more organic acids from plant cells, particularly from the roots. Examples of these organic acids include, but are not limited to, citrate, oxalate and malate. In a particular embodiment, the organic acid is malate.

In a further embodiment, the polypeptide having aluminium activated organic acid transporter activity comprises;

a) an amino acid sequence as provided in SEQ ID NO:1;

b) an amino acid sequence as provided in SEQ-ID NO:3;

c) an amino acid sequence as provided in SEQ ID NO:24;

d) an amino acid sequence as provided in SEQ ID NO:26;

e) an amino acid sequence which is at least 50% identical to any one of a) to d), or

f) an amino acid sequence which is a biologically active fragment of any one of a) to e).

In further embodiments, the polypeptide comprises an amino acid sequence which is at least 60%, at least 65%, at least 70%, at least 75%, at least 76%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6 at least 99.7%, at least 99.8%, or at least 99.9% identical to SEQ ID NO:1, SEQ ID NO:3, SEQ ID NO:24 or SEQ ID NO:26.

In another embodiment, the polypeptide having aluminium activated organic acid transporter activity is encoded by a polynucleotide comprising;

a) a nucleotide sequence as provided in SEQ ID NO:2;

b) a nucleotide sequence as provided in SEQ ID NO:4;

c) a nucleotide sequence as provided in SEQ ID NO:23;

d) a nucleotide sequence as provided in SEQ ID NO:25;

e) a nucleotide sequence which is at least 50% identical to any one of a) to d),

f) a nucleotide sequence which hybridizes to any one of a) to d) under stringent conditions, or

g) a nucleotide sequence which is a fragment of one of a) to f) encoding a polypeptide having aluminium activated organic acid transporter activity.

In one embodiment, the polynucleotide of parts e) and/or f) comprise a nucleotide sequence as provided in SEQ ID NO:6.

In other embodiments, the polynucleotide comprises a nucleotide sequence which is at least 60%, at least 65%, at least 70%, at least 75%, at least 76%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to SEQ ID NO:2, SEQ ID NO:4, SEQ ID NO:23 or SEQ ID NO:25.

In another embodiment, the root apices of the barley plant of the invention do not stain with hematoxylin or stain substantially less than wild-type roots. Such staining procedures can be performed as typically used in the art. Hematoxylin staining may be performed as described in the Examples section herein or in Echart et al. (2002). Typically, roots are washed with deionised or distilled water, then immersed in 0.2% (w/v) hematoxylin/0.02% (w/v) KIO3 for 30 min at room temperature.

In a further aspect, the present invention provides a method of producing a barley plant comprising an exogenous nucleic acid molecule and having enhanced tolerance to aluminium relative to an isogenic plant not comprising said exogenous nucleic acid molecule, the method comprising;

a) introducing at least one exogenous nucleic acid molecule into at least one barley cell, wherein said nucleic acid molecule comprises a regulatory element operably linked to a polynucleotide encoding a polypeptide that confers enhanced tolerance to aluminium to a barley plant,

b) obtaining one or more plants from said cell; and

c) identifying at least one of said plants that has enhanced tolerance to aluminium relative to an isogenic plant not comprising said exogenous nucleic acid molecule.

In a further embodiment, the method further comprises a step of producing a plant line from said at least one plant of step c) by self- or cross-pollination.

In another embodiment, the polypeptide that confers enhanced tolerance to aluminium comprises;

a) an amino acid sequence as provided in SEQ ID NO:1;

b) an amino acid sequence as provided in SEQ ID NO:3;

c) an amino acid sequence as provided in SEQ ID NO:24;

d) an amino acid sequence as provided in SEQ ID NO:26;

e) an amino acid sequence which is at least 50% identical to any one of a) to d), or

f) an amino acid sequence which is a biologically active fragment of any one of a) to e).

In further embodiments, the polypeptide comprises an amino acid sequence which is at least 60%, at least 65%, at least 70%, at least 75%, at least 76%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to SEQ ID NO: 1, SEQ ID NO:3, SEQ ID NO:24 or SEQ ID NO:26.

The regulatory element can be any suitable polynucleotide which is capable of directing gene expression in a barley cell. Thus, the regulatory element may be capable of directing expression of the polynucleotide in all cells of the barley plant, or a subset of cells. However, it is preferred that the regulatory element at least directs expression of the polynucleotide in cells of the root apices.

In a further embodiment, the regulatory element is a constitutive promoter. In a particular embodiment, the promoter is a ubiquitin promoter.

The exogenous nucleic acid molecule may also comprise a polyadenylation signal 3′ of said polynucleotide.

Also provided is a barley plant produced by a method of the invention.

The barley plants of the invention are useful for grain production, in particular for commercial grain production. The desired genetic background of the barley will include considerations of agronomic yield and other characteristics. Such characteristics might include whether it is desired to have a winter or spring type of barley, agronomic performance, disease resistance and abiotic stress resistance. It would be readily understood that the exogenous nucleic acid molecule providing the aluminium tolerance trait can be combined with other useful genetic traits by conventional breeding, using a plant of the invention as a parent in crossing.

Thus, the present invention also provides a method of producing grain, the method comprising;

a) growing a barley plant according to the invention, and

b) harvesting the grain.

Preferably, the plant is grown in an acidic soil.

In a further aspect, the present invention relates to grain from the barley plant of the invention.

In another aspect, the present invention provides a method of producing flour, wholemeal, starch or malt, the method comprising;

a) obtaining grain according to the invention, and

b) extracting the flour, wholemeal, or starch, or

c) malting the grain.

Such methods are well known to those skilled in the art.

In a further embodiment, the invention provides a milled product derived from grain including, but not limited to, flour, wholemeal, starch or malt obtained from the grain of the invention, or food or drink products incorporating such flour, wholemeal, starch or malt, or rolled, flaked or extruded products of the grain. The product may be blended with flour, wholemeal, starch or malt from another source. Additionally, the invention encompasses grain that has been processed in other ways, so that the grain may have been, for example, milled, ground, rolled, pearled, kibbled or cracked.

In yet a further aspect, the present invention provides a substantially purified polypeptide selected from:

a) a polypeptide comprising an amino acid sequence as provided in SEQ ID NO:24,

b) a polypeptide comprising an amino acid sequence as provided in SEQ ID NO:26,

c) a polypeptide comprising an amino acid sequence which is at least 70% identical to a) or b), and

d) a biologically active fragment of a) or b),

wherein the polypeptide has aluminium activated organic acid transporter activity.

In one embodiment, the polypeptide can be purified from a species of the Genus Hordeum.

In a further embodiment, the polypeptide is a fusion protein further comprising at least one other polypeptide sequence.

In a preferred embodiment, the at least one other polypeptide is selected from: a polypeptide that enhances the stability of a polypeptide of the present invention, or a polypeptide that assists in the purification of the fusion protein.

In a further aspect, the present invention provides an isolated polynucleotide comprising a sequence of nucleotides selected from:

a) a sequence of nucleotides as provided in SEQ ID NO:23;

b) a sequence of nucleotides as provided in SEQ ID NO:25;

c) a sequence of nucleotides as provided in SEQ ID NO:22;

d) a sequence of nucleotides encoding a polypeptide of the invention;

e) a sequence of nucleotides which is at least 75% identical to any one of a) to c); and

f) a sequence which hybridizes to any one of a) to e) under stringent conditions,

wherein the polynucleotide does not consist of a sequence of nucleotides as provided in SEQ ID NO:5.

In a preferred embodiment, the polynucleotide is at least 630 nucleotides in length. In a further preferred embodiment, the polynucleotide encodes a polypeptide having aluminium activated organic acid transporter activity.

In a further aspect, the present invention provides a vector comprising a polynucleotide of the invention.

In yet another aspect, the present invention provides a host cell comprising a vector of the invention, and/or an isolated polynucleotide of the invention.

Preferably, the host cell is a plant cell, more preferably a barley cell.

In another aspect, the present invention provides a substantially purified antibody, or fragment thereof, that specifically binds a polypeptide comprising an amino acid sequence as provided in SEQ ID NO:24 or SEQ ID NO:26, wherein the antibody does not bind a polypeptide provided as SEQ ID NO:1 or SEQ ID NO:3.

Preferably, the antibody is detectably labelled.

In yet a further aspect, the present invention provides a transgenic plant comprising an exogenous nucleic acid molecule, the nucleic acid molecule encoding a polypeptide of the invention. In this aspect, the transgenic plant can be of any species. Preferably, the plant is a cereal plant such as wheat or barley. Methods for producing such transgenic plants are well known in the art.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying figures.

### BRIEF DESCRIPTION OF THE ACCOMPANYING DRAWINGS

FIG. 1—Expression levels of ALMT1 in primary transformants of barley. Real time quantitative RT-PCR was used to assess ALMT1 mRNA expression levels in root apices of three independent transgenic barley lines (lines 4, 5 and 6) transformed with the ALMT1 coding region, and the near-isogenic wheat lines ET8/ES8 that differ in Al tolerance (ET8: tolerant; ES8: susceptible). The controls consisted of non-transformed wild-type plants (WT) and plants transformed with the vector without ALMT1 (Vec8). Expression was assessed relative to the expression level of a phosphate transporter gene (black columns) or a proton pump control gene (grey columns), and is shown standardized to the highest expressing transgenic line (arbitrarily set to 1.0). The bars show ±standard errors of the means (n=3).

FIG. 2—Southern blot hybridization analysis of the transgenic barley lines expressing ALMT1 (lines 4 to 6) and control plants (WT: wild type; Vec8: plasmid control). Genomic DNA was digested with HindIII and the filter probed with the ALMT1 coding region. The band in common in all lanes corresponds to a sequence in barley that is related to ALMT1.

FIG. 3—ALMT1 expression confers Al-activated malate efflux from barley roots.
(A) Al-activated malate efflux from root apices of transgenic barley expressing ALMT1 and control lines (WT and Vec8). Excised root apices were incubated with 50 μM Al solution in basal solution (0.2 mM CaCl₂ pH 4.3) and solutions were changed after every hour. Error bars show ±standard errors of the means (n=3). Excised root apices incubated in basal solution only exuded little or no malate for all genotypes.
(B) Al-activated malate efflux from root segments of transgenic barley line 5 expressing ALMT1. The measurements indicate the distance from the end of the root apex. Excised root segments were incubated for 1 h with 50 μM Al solution in basal solution (0.2 mM CaCl₂ pH 4.3; black columns) or in basal solution only (grey columns). Error bars show ±standard errors of the means (n=3).
(C) Relative ALMT1 expression in root segments of transgenic barley line 5. RNA from excised root segments was extracted, transcribed to cDNA and analyzed for ALMT1 expression by real time quantitative RT-PCR. Expression is expressed relative to phosphate transporter (black columns) or proton pump (grey columns) genes and is shown as a proportion of the 0 to 3 mm segment (arbitrarily set to 1.0). Error bars show ±standard errors of the means (n=3).

FIG. 4—Properties of malate efflux from root apices of transgenic barley expressing ALMT1.
(A) Effect of Al concentration on malate efflux (nmoles/h/apex). Excised root apices from ALMT1 line 5 (solid circles) were exposed to Al treatments from 0 to 50 μM in basal solution for 1 h. Error bars show ±standard errors of the means (n=3). The solid squares show efflux from root apices of the control line transformed with the empty vector.
(B) Effects of niflumic acid (nif; 0 to 100 μM), La (50 μM), Er (50 μM) and basal solution (0.2 mM CaCl₂ pH 4.3) on malate efflux from root apices of line 5. Excised root apices from line 5 were exposed to the various treatments in basal solution for 1 h. Error bars show ±standard errors of the means (n=3).

FIG. 5—ALMT1 confers Al tolerance to barley grown in hydroponic culture.
(A) Root elongation of T0 (primary transgenics) barley lines grown in hydroponic culture. Plants were grown for 10 d in nutrient solution that contained a range of Al concentrations from 0 to 12 μM. Control lines included wild type (WT) and a transgenic line transformed with the vector only (Vec8). Root elongation over this period is expressed as a percentage of the elongation in the absence of Al. Error bars show ±standard errors of the means (n=4). Root lengths of plants grown in the absence of Al were as follows: wild type: 103±14 mm; vector only control: 103±14 mm; ALMT1 line 4: 99±12 mm; ALMT1 line 5: 122±12 mm; and ALMT1 line 6: 118±8 mm.
(B) Effect of 3 μM Al on growth over 10 d of the T0 generation of the control (Vec8) line (plant on right) and ALMT1 line 5 (plant on left).

FIG. 6—Scanning electron micrograph showing the effect of Al (3 μM) on the morphology of the root apex from the control line (Vec8) and ALMT1 line 5 grown for 10 d.

FIG. 7—Root elongation of T2 homozygous barley lines grown in hydroponic culture. For each transgenic line a sister line azygous for ALMT1 derived from the same transformation event was developed. The wheat lines ET8 and ES8 are near isogenic lines that differ in Al tolerance. Elongation of the longest root over 5 d is expressed as a percentage of the minus Al treatment and error bars show ±standard errors of the means (n=7). Root lengths of plants grown in the absence of Al were as follows: wild type: 54±3 mm; ALMT1 line 4: 62±4 mm; azygous line 4: 57±4 mm; ALMT1 line 5: 46±3 mm; azygous line 5: 60±3 mm; ALMT1 line 6: 60±4 mm; azygous line 6: 63±1 mm; ET8: 66±3 mm and ES8 74±4 mm.

FIG. 8—ALMT1 enhances root growth of barley on acid soils.
A. Seedlings of the various T2 homozygous ALMT1-expressing lines of barley, their azygous sister lines, wild type barley (WT), and the near-isogenic wheat lines ET8/ES8 were grown on an un-amended acid soil (“Acid soil”) obtained from Chiltern, Australia and the same soil neutralized with CaCO₃ (“Neutral soil”). After 4 d, the longest root of each seedling was measured and root growth over 4 d calculated (error bars denote ±standard errors of means [n=6 to 8]; and the least significant difference [LSD] at P<0.05 is shown for the interactions of root length with treatments and genotypes).
B. Seedlings of the various T2 homozygous ALMT1-expressing lines of barley, wild type barley (WT), and the near-isogenic wheat lines ET8/ES8 were grown on an acid soil obtained from Tohoku in Japan and a neutral medium consisting of fertilized peat-moss. After 4 d the longest root of each seedling was measured (error bars denote a standard errors of means [n=6]; and the least significant difference [LSD] at P<0.05 is shown for the interactions of root length with treatments and genotypes).

FIG. 9—Alignment of nucleotide sequences (cDNA) of the wheat ALMT1 protein coding region (ALMT-1coding) (SEQ ID NO:2), a barley EST sequence (HvALMT1HomoSprime) (SEQ ID NO:5) and a rice homolog protein coding region (OsALMT1RiceCoding) (SEQ ID NO:6). Conserved nucleotides are shaded.

FIG. 10—Phylogenetic relationships of the ALMT1 protein and Arabidopsis homologues. The Arabidopsis gene designation and the distance to the branch point for each sequence are indicated.

FIG. 11—Genomic sequence of the barley cv Morex HvALMT1 ortholog. Putative exons are shown bold and introns as normal text. Positions of exons: 1-331; 440-580; 808-1080; 1445-1557; 1683-1832; 1912-2271 (SEQ ID NO:22).

FIG. 12—Alignment of wheat ALMT1-1 coding region (SEQ ID NO:2) to the barley (cv Dayton) ortholog nucleic acid sequence (SEQ ID NO:23).

FIG. 13—Alignment of wheat ALMT1-1 amino acid sequence (SEQ ID NO:1) to the barley (cv Dayton) ortholog amino acid sequence (SEQ ID NO:24).

FIG. 14—Alignment of barley EST (SEQ ID NO:5) (Accession No. BU993212) with the Dayton HvALMT1 coding sequence (SEQ ID NO:23).

FIG. 15—Relative aluminium tolerance of wild-type Arabidopsis ecotype Columbia-0 (WT) and mutant Line #10. The two longest roots on plants of each population were measured after 8 d growth on agar medium containing nutrients and a range of AlCl₃ concentrations. Upper graph shows absolute root length measurements and the lower graph shows relative root growth as a percentage of the growth in controls. Data show mean and SD (n=11-22).

FIG. 16—Relative aluminium tolerance of wild-type Arabidopsis ecotype Columbia-0 (Col WT) and mutant Line #9. The two longest roots on plants of each population were measured after 14 d growth on agar medium containing nutrients and a range of AlCl₃ concentrations. Upper graph shows absolute root length measurements and the lower graph shows relative root growth as a percentage of the growth in controls. Data show mean and SD (n=15-18).

FIG. 17—Nucleotide (SEQ ID NO:7) and amino acid (SEQ ID NO:3) sequences of the Arabidopsis At1g08430 gene. The coding region corresponds to nucleotides 78-1559 of the nucleotide sequence.

FIG. 18—Overexpression of Atg08430 confers Al tolerance to Arabidopsis ecotype Columbia The Al tolerance of Arabidopsis plants overexpressing Atg08430 was compared to wild-type. Seed was sown on agar medium supplemented with low-ionic strength mineral nutrients at pH 4.5 and having various concentrations of AlCl₃ added. After 15 d growth under illumination, root length was measured. The top figure shows mean root lengths with error bars denoting one standard deviation. The bottom figure shows the data expressed as a percentage of the zero Al treatment.

FIG. 19—Southern blot of DNA extracted from several plant species and probed with a fragment of the Arabidopsis gene At1g08430. Lane 1—size standard (1.65 kb); Lane 2—Arabidopsis; Lane 3—Brassica napus; Lane 4—Brassica juncea; Lane 5—wheat (ET8); Lane 6—wheat (ES8); Lane 7—Trifolium subterranean (1); Trifolium subterranean line 2; Lane 9—Arabidopsis; Lane 10—size standard (1.65 kb).

FIG. 20—Malate efflux from whole roots measured over 2 h intervals, for transgenic tobacco plants expressing wheat ALMT1 compared to control plants. At each time point the malate accumulated in the solution was assayed and the solution replaced. The means and error bars denoting one standard deviation (n=3) are shown.

### KEY TO THE SEQUENCE LISTING

SEQ ID NO:1—ALMT1 protein from Triticum aestivum.
SEQ ID NO:2—Open reading frame encoding the ALMT1 protein from Triticum aestivum.
SEQ ID NO:3—At1g08430 protein (homolog of wheat ALMT1) from Arabidopsis thaliana.
SEQ ID NO:4—Open reading frame encoding At1g08430 protein from Arabidopsis thaliana.
SEQ ID NO:5—EST (Accession No. BU993212) from Hordeum vulgare encoding homolog of wheat ALMT1.
SEQ ID NO:6—Open reading frame encoding Ozyza sativa homolog of wheat ALMT1.
SEQ ID NO:7—cDNA sequence of SEQ ID NO:2 comprising additional 5′ sequence (FIG. 17).
SEQ ID NO's 8 to 21, 27 and 28—Oligonucleotide primers.
SEQ ID NO:22—Genomic sequence of barley cv Morex HvALMT1.
SEQ ID NO:23—cDNA sequence encoding barley cv Dayton HvALMT1.
SEQ ID NO:24—Protein sequence of barley cv Dayton HvALMT1.
SEQ ID NO:25—cDNA sequence encoding barley cv Morex HvALMT1.
SEQ ID NO:26—Protein sequence of barley cv Morex HvALMT1.

### DETAILED DESCRIPTION OF THE INVENTION

### General Techniques

Unless specifically defined otherwise, all technical and scientific terms used herein shall be taken to have the same meaning as commonly understood by one of ordinary skill in the art (e.g., in cell culture, molecular genetics, plant physiology, plant biochemistry, plant breeding, immunohistochemistry and protein chemistry).

Unless otherwise indicated, the recombinant protein, cell culture and others methods utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hamines (editors), DNA Cloning: A Practical Approach, Volumes 1-4, IRL Press (1995 and 1996), and F. M. Ausubel et al., (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al., (editors) Current Protocols in Immunology, John Wiley & Sons (including all updates until present), and are incorporated herein by reference.

### SELECTED DEFINITIONS

As used herein, the term “barley” refers to any species of the Genus Hordeum, including progenitors thereof, as well as progeny thereof produced by crosses with other species. It is preferred that the plant is of a Hordeum species which is commercially cultivated such as, for example, a strain or cultivar or variety of Hordeum vulgare.

The phrase “enhanced tolerance to aluminium” is used herein as a relative term to describe an increased tolerance when the plant is grown in the presence of aluminium relative to a wild-type barley plant. The best comparison is with an isogenic barley plant that is lacking the exogenous nucleic acid molecule. Typically, the portion of the plant that is exposed to aluminium is the roots, with the apex of the roots being most sensitive to aluminium. As a result, indicators of enhanced tolerance to aluminium include increased root growth, and/or increased secretion of an organic acid (such as, for example, malate) by cells of the root apices, when the plant is grown in soils high in aluminium (namely, which include at least about 1 μM of the solubilized trivalent cation Al³⁺) such as found in acidic soils. However, other indicators of enhanced tolerance to aluminium are the overall health of the plant including, but not limited to, increased grain production when the plant is grown in soils high in aluminium, increased biomass, shoot height, increased leaf area, number of tillers per plant, decreased leaf damage or senescence and the like.

The phrase “without significant inhibition of root growth” is used herein as a relative term to describe the amount or rate of root growth relative to growth in the absence (or near absence) of aluminium. Root growth may be measured in terms of the length, dry weight, content of protein or other compounds, or the like. Measurement of the length is a simple, non-destructive means of determining root growth. Root growth can be examined in the “screening solution” such as, for example, a hydroponic growth solution, defined herein with and without the presence of aluminium chloride. In certain embodiments, roots of a barley plant of the invention grown in the presence of at least about 5 μM aluminium are (on average) at least 50%, at least 80% or at least 90% the length of the roots of a plant of the same genotype (including any transgenes) grown in the absence of aluminium.

As used herein, an acidic soil has a pH of less than 5.8, a “moderately acidic soil” has a pH less than 5.5, and a “highly acid soil” has a pH of less than 5.2. The growth of wild-type barley plants is affected at pH 5.5 or less, and seriously affected at pH below 5.2.

As used herein, the term “aluminium activated organic acid transporter activity” refers to proteins which, upon-exposure to aluminium, cause a cell to secrete an organic acid such as, but not limited to, citrate, oxalate or malate. The term “aluminium activated” refers to the property that the rate or amount of organic acid that is secreted is increased by at least 50% by the presence of aluminium chloride at a level of at least 5 μM. In one embodiment, the aluminium activated organic acid transporter useful for the plants or methods of the invention is an ALMT1 protein of barley or wheat, or homologs from other plant species such as the At1g08430 protein from Arabidopsis thaliana.

The term “plant” includes whole plants, vegetative structures (for example, leaves, stems), roots, floral organs/structures, seed (including embryo, endosperm, and seed coat), plant tissue (for example, vascular tissue, ground tissue, and the like), cells and progeny of the same.

A “transgenic plant” refers to a plant that contains a gene construct (“transgene”) not found in a wild-type plant of the same species, variety or cultivar. A “transgene” as referred to herein has the normal meaning in the art of biotechnology and includes a genetic sequence which has been produced or altered by recombinant DNA or RNA technology and which has been introduced into the barley cell. The transgene may include genetic sequences derived from a barley cell. Typically, the transgene has been introduced into the plant by human manipulation such as, for example, by transformation but any method can be used as one of skill in the art recognizes.

As used herein, the term “root apex”, or the plural “root apices”, refers to a region of the root spanning about 5 mm, more preferably about 3 mm, of the root tip.

“Wild type”, as used herein, refers to a cell, tissue or plant that has not been modified according to the invention. Wild-type cells, tissue or plants may be used as controls to compare levels of expression of the exogenous nucleic acid molecule or the extent and nature of trait modification with cells, tissue or plants modified as described herein.

The term “isogenic” refers to cell, tissue or plant that that has the same genotype as a cell, tissue or plant of the invention but without the exogenous nucleic acid molecule which confers enhanced tolerance to aluminium. Typically, the wild-type plant will be a non-transgenic barley plant of the same variety or cultivar as the plant into which the exogenous nucleic acid molecule was introduced. Plants isogenic to those of the invention can be used as controls to compare levels of exogenous nucleic acid expression or the extent and nature of trait modification with cells, tissue or plants modified as described herein.

“Nucleic acid molecule” refers to a oligonucleotide, polynucleotide or any fragment thereof. It may be DNA or RNA of genomic or synthetic origin, double-stranded or single-stranded, and combined with carbohydrate, lipids, protein, or other materials to perform a particular activity defined herein.

“Operably linked” as used herein refers to a functional relationship between two or more nucleic acid (e.g., DNA) segments. Typically, it refers to the functional relationship of transcriptional regulatory element to a transcribed sequence. For example, a promoter is operably linked to a coding sequence, such as a polynucleotide defined herein, if it stimulates or modulates the transcription of the coding sequence in an appropriate barley cell. Generally, promoter transcriptional regulatory elements that are operably linked to a transcribed sequence are physically contiguous to the transcribed sequence, i.e., they are cis-acting. However, some transcriptional regulatory elements, such as enhancers, need not be physically contiguous or located in close proximity to the coding sequences whose transcription they enhance.

As used herein, the term “gene” is to be taken in its broadest context and includes the deoxyribonucleotide sequences comprising the protein coding region of a structural gene and including sequences located adjacent to the coding region on both the 5′ and 3′ ends for a distance of about 1 kb on either end. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region which may be interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide. The term “gene” includes a synthetic or fusion molecule encoding all or part of the proteins of the invention described herein and a complementary nucleotide sequence to any one of the above.

In reference to nucleic acid sequences which “correspond” to a gene, the term “correspond” refers to a nucleotide sequence relationship, such that the nucleotide sequence has a nucleotide sequence which is the same as the reference gene or an indicated portion thereof, or has a nucleotide sequence which is exactly complementary in normal Watson-Crick base pairing, or is an RNA equivalent of such a sequence, for example, an mRNA, or is a cDNA derived from an mRNA of the gene.

When present in a cell, preferably a barley cell, a “gene” directs the “expression” of a “biologically active” molecule or “gene product”, which may be RNA or a polypeptide. This process is most commonly by transcription to produce RNA and translation to produce protein. Such a product may be subsequently modified in the cell. RNA may be modified by, for example, polyadenylation, splicing, capping, dicing into 21-23 nucleotide fragments, or export from the nucleus or by covalent or noncovalent interactions with proteins. Proteins may be modified by, for example, phosphorylation, glycosylation or lipidation. All of these processes are encompassed by the term “expression of a gene” or the like as used herein.

### Nucleic Acid Constructs

Exogenous nucleic acid molecules of the invention encode a polypeptide that confers enhanced tolerance to aluminium to a barley plant cell. In a preferred embodiment, the encoded polypeptide possesses aluminium activated organic acid transporter activity. The nucleic acid constructs may comprise intron sequences. These intron sequences may aid expression of the transgene in barley plants. The term “intron” is used in its normal sense as meaning a genetic segment that is transcribed but does not encode protein and which is spliced out of an RNA before translation. Introns may be incorporated in a 5′-UTR or a coding region if the transgene encodes a translated product, or anywhere in the transcribed region if it does not. However, in a preferred embodiment, the polypeptide is encoded by a single open reading frame. As the skilled addressee would be aware, such open reading frames can be obtained by reverse transcribing mRNA encoding the polypeptide.

To ensure appropriate expression of the gene encoding the polypeptide that confers enhanced tolerance to aluminium to a barley plant cell, the nucleic acid construct typically comprises one or more regulatory elements such as promoters, enhancers, as well as transcription termination or polyadenylation sequences. Such elements are well known in the art.

The transcriptional initiation region comprising the regulatory element(s) may provide for regulated or constitutive expression in the barley plant. Preferably, expression at least occurs in cells of the root apices. The regulatory elements may be selected be from, for example, root-specific promoters, or promoters not specific for root cells (such as ubiquitin promoter or CaMV35S or enhanced 35S promoters). The promoter may be modulated by factors such as temperature, light or stress. Ordinarily, the regulatory elements will be provided 5′ of the genetic sequence to be expressed. The construct may also contain other elements that enhance transcription such as the nos 3′ or the ocs 3′ polyadenylation regions or transcription terminators.

Typically, the nucleic acid construct comprises a selectable marker. Selectable markers aid in the identification and screening of plants or cells that have been transformed with the exogenous nucleic acid molecule. The selectable marker gene may provide antibiotic or herbicide resistance to the barley cells, or allow the utilization of substrates such as mannose. The selectable marker preferably confers hygromycin resistance to the barley cells.

Preferably, the nucleic acid construct is stably incorporated into the genome of the barley plant. Accordingly, the nucleic acid comprises appropriate elements which allow the molecule to be incorporated into the barely genome, or the construct is placed in an appropriate vector which can be incorporated into a chromosome of a barley cell.

### Transformation Methods for Barley

Methods for transformation of monocotyledonous plants such as barley for introducing genetic variation into the plant by introduction of an exogenous nucleic acid and for regeneration of plants from protoplasts or immature plant embryos are well known in the art, see for example, Wan and Lemaux (1994), Tingay et al (1997), Canadian Patent Application No. 2,092,588, Australian Patent Application No 61781/94, Australian Patent No 667939, U.S. Pat. No. 6,100,447, International Patent Application PCT/US97/10621, U.S. Pat. No. 5,589,617, U.S. Pat. No. 6,541,257, and other methods are set out in Patent specification WO99/14314, as well as those described in the Examples section herein. Preferably, transgenic barley are produced by Agrobacterium tumefaciens mediated transformation procedures. Vectors carrying the desired nucleic acid construct may be introduced into regenerable barley cells of tissue cultured plants or explants, or suitable plant systems such as protoplasts.

The regenerable barley cells are preferably from the scutellum of immature embryos, mature embryos, callus derived from these, or the meristematic tissue.

Any of several methods may be employed to determine the presence of a transformed plant. For example, polymerase chain reaction (PCR) may be used to amplify sequences that are unique to the transformed plant, with detection of the amplified products by gel electrophoresis or other methods. DNA may be extracted from the plants using conventional methods and the PCR reaction carried out using primers that will distinguish the transformed and non-transformed plants. For example, primers may be designed that will amplify a region of DNA from the transformation vector reading into the construct and the reverse primer designed from the gene of interest. These primers will only amplify a fragment if the plant has been successfully transformed. An alternative method to confirm a positive transformant is by Southern blot hybridization, well known in the art. Plants which are transformed may also be identified i.e. distinguished from non-transformed or wild-type plants by their phenotype, for example conferred by the presence of a selectable marker gene, or conferred by the activity of a polypeptide that provides enhanced tolerance to aluminium.

### Polypeptides

By “substantially purified polypeptide” we mean a polypeptide that has been at least partially separated from the lipids, nucleic acids, other polypeptides, and other contaminating molecules with which it is associated in its native state. Preferably, the substantially purified polypeptide is at least 60% free, preferably at least 75% free, and most preferably at least 90% free from other components with which they are naturally associated. Furthermore, the term “polypeptide” is used interchangeably herein with the term “protein”:

The % identity of a polypeptide is determined by GAP (Needleman and Wunsch, 1970) analysis (GCG program) with a gap creation penalty=5, and a gap extension penalty=0.3. Unless stated otherwise, the query sequence is at least 15 amino acids in length, and the GAP analysis aligns the two sequences over a region of at least amino acids. More preferably, the query sequence is at least 50 amino acids in length, and the GAP analysis aligns the two sequences over a region of at least 50 amino acids. Even more preferably, the query sequence is at least 100 amino acids in length and the GAP analysis aligns the two sequences over a region of at least 100 amino acids.

With regard to the aspects of the invention which relate to a substantially purified polypeptide, it will be appreciated that % identity figures higher than those provided above will encompass preferred embodiments. Thus, it is preferred that the polypeptide comprises an amino acid sequence which is at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to SEQ ID NO:24 or SEQ ID NO:26.

A “biologically active fragment” of a polypeptide defined herein is a molecule that has had portion of the full length molecule removed, typically at the N- and/or C-terminus, but still maintains at least some of the activity of the full length protein. In the context of the present invention, the activity is the ability to confer upon a barley plant an enhanced tolerance to aluminium.

Amino acid sequence mutants of naturally occurring polypeptides which have aluminium activated organic acid transporter activity (for example, SEQ ID NO:1, SEQ ID NO:3, SEQ ID NO:24 and SEQ ID NO:26) can be prepared by introducing appropriate nucleotide changes into a polynucleotide defined herein (for example SEQ ID NO:2, SEQ ID NO:4 SEQ ID NO:23 or SEQ ID NO:25 respectively), or by in vitro synthesis of the desired polypeptide. Such mutants include, for example, deletions, insertions or substitutions of residues within the amino acid sequence. A combination of deletion, insertion and substitution can be made to arrive at the final construct, provided that the final protein product possesses the desired characteristics.

Amino acid sequence deletions generally range from about 1 to 30 residues, more preferably about 1 to 10 residues and typically about 1 to 5 contiguous residues.

Substitution mutants have at least one amino acid residue in the polypeptide molecule removed and a different residue inserted in its place. The sites of greatest interest for substitutional mutagenesis include sites identified as the active or binding site(s). Other sites of interest are those in which particular residues obtained from various strains or species are identical. These positions may be important for biological activity. These sites, especially those falling within a sequence of at least three other identically conserved sites, are preferably substituted in a relatively conservative manner. Such conservative substitutions are shown in Table 1.

Furthermore, if desired, unnatural amino acids or chemical amino acid analogues can be introduced as a substitution or addition into the polypeptides of the present invention. Such amino acids include, but are not limited to, the D-isomers of the common amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogues in general.

Also included within the scope of the invention are polypeptides of the present invention which are differentially modified during or after synthesis, e.g., by biotinylation, benzylation, glycosylation, acetylation, phosphorylation, amidation, derivatization by known protecting/blocking groups, proteolytic cleavage, linkage to an antibody molecule or other cellular ligand, etc. These modifications may serve to increase the stability and/or bioactivity of the polypeptide of the invention.

TABLE 1
Exemplary substitutions.

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe, ala |

Polypeptides of the present invention can be produced in a variety of ways, including production and recovery of natural proteins, production and recovery of recombinant proteins, and chemical synthesis of the proteins. In one embodiment, an isolated polypeptide of the present invention is produced by culturing a cell capable of expressing the polypeptide under conditions effective to produce the polypeptide, and recovering the polypeptide. A preferred cell to culture is a recombinant cell of the present invention. Effective culture conditions include, but are not limited to, effective media, bioreactor, temperature, pH and oxygen conditions that permit protein production. An effective medium refers to any medium in which a cell is cultured to produce a polypeptide of the present invention. Such medium typically comprises an aqueous medium having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

### Polynucleotides

By an “isolated polynucleotide”, including DNA, RNA, or a combination of these, single or double stranded, in the sense or antisense orientation or a combination of both, dsRNA or otherwise, we mean a polynucleotide which is at least partially separated from the polynucleotide sequences with which it is associated or linked in its native state. Preferably, the isolated polynucleotide is at least 60% free, preferably at least 75% free, and most preferably at least 90% free from other components with which they are naturally associated. Furthermore, the term “polynucleotide” is used interchangeably herein with the term “nucleic acid molecule”.

The % identity of a polynucleotide is determined by GAP (Needleman and Wunsch, 1970) analysis (GCG program) with a gap creation penalty=5, and a gap extension penalty 0.3. Unless stated otherwise, the query sequence is at least 45 nucleotides in length, and the GAP analysis aligns the two sequences over a region of at least 45 nucleotides. Preferably, the query sequence is at least 150 nucleotides in length, and the GAP analysis aligns the two sequences over a region of at least 150 nucleotides. More preferably, the query sequence is at least 300 nucleotides in length and the GAP analysis aligns the two sequences over a region of at least 300 nucleotides.

With regard to the aspects of the invention which relate to an isolated polynucleotide, it will be appreciated that % identity figures higher than those provided above will encompass preferred embodiments. Thus, it is preferred that the polypeptide comprises an amino acid sequence which is at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to SEQ ID NO:23 or SEQ ID NO:25.

Polynucleotides of the invention, and polynucleotides useful for the production of transgenic barley of the present invention, include those which hybridize under stringent conditions to a sequence provided as, for example, SEQ ID NO:2 and/or SEQ ID NO:4. As used herein, stringent conditions are those that (1) employ low ionic strength and high temperature for washing, for example, 0.015 M NaCl/0.0015 M sodium citrate/0.1% NaDodSO₄ at 50° C.; (2) employ during hybridisation a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS and 10% dextran sulfate at 42° C. in 0.2×SSC and 0.1% SDS.

Generally, a naturally occurring gene useful for the production of barley of the invention may be subjected to mutagenesis to produce single or multiple nucleotide substitutions, deletions and/or additions such as, for example, codon modification. Nucleotide insertional derivatives of such genes include 5′ and 3′ terminal fusions as well as intra-sequence insertions of single or multiple nucleotides. Insertional nucleotide sequence variants are those in which one or more nucleotides are introduced into a predetermined site in the nucleotide sequence, although random insertion is also possible with suitable screening of the resulting product. Deletional variants are characterised by the removal of one or more nucleotides, from the sequence.

Substitutional nucleotide variants are those in which at least one nucleotide in the sequence has been removed and a different nucleotide inserted in its place. Such a substitution may be “silent” in that the substitution does not change the amino acid defined by the codon. Alternatively, conservative substituents are designed to alter one amino acid for another similar acting amino acid.

### Vectors

One embodiment of the present invention includes a recombinant vector, which includes at least one isolated polynucleotide molecule of the present invention inserted into any vector capable of delivering the nucleic acid molecule into a host cell. Such a vector contains heterologous nucleic acid sequences, that is nucleic acid sequences that are not naturally found adjacent to nucleic acid molecules of the present invention and that preferably are derived from a species other than the species from which the nucleic acid molecule(s) are derived. The vector can be either RNA or DNA, either prokaryotic or eukaryotic, and typically is a virus or a plasmid.

One type of recombinant vector comprises a nucleic acid molecule of the present invention operatively linked to an expression vector. The phrase operatively linked refers to insertion of a nucleic acid molecule into an expression vector in a manner such that the molecule is able to be expressed when transformed into a host cell. As used herein, an expression vector is a DNA or RNA vector that is capable of transforming a host cell and effecting expression of a specified nucleic acid molecule. Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in recombinant cells of the present invention, including in bacterial, fungal, endoparasite, arthropod, other animal, and plant cells. Preferred expression vectors of the present invention can direct gene expression in plant cells.

In particular, expression vectors of the present invention contain regulatory sequences such as transcription control sequences, translation control sequences, origins of replication, and other regulatory sequences that are compatible with the recombinant cell and that control the expression of nucleic acid molecules of the present invention. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art.

### Host Cells

Another embodiment of the present invention includes a recombinant cell comprising a host cell transformed with one or more recombinant molecules of the present invention. Transformation of a nucleic acid molecule into a cell can be accomplished by any method by which a nucleic acid molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed nucleic acid molecules of the present invention can remain extrachromosomal or can integrate into one or more sites within a chromosome of the transformed (i.e., recombinant) cell in such a manner that their ability to be expressed is retained.

Suitable host cells to transform include any cell that can be transformed with a polynucleotide of the present invention. Host cells can be either untransformed cells, or cells that are already transformed with at least one nucleic acid molecule (e.g., nucleic acid molecules encoding one or more proteins of the present invention). Host cells of the present invention either can be endogenously (i.e., naturally) capable of producing proteins of the present invention or can be capable of producing such proteins after being transformed with at least one nucleic acid molecule of the present invention. Host cells of the present invention can be any cell capable of producing at least one protein of the present invention, and include bacterial, fungal (including yeast), parasite, arthropod, animal and plant cells. Preferred host cells are plant cells, in particular barley cells. In a preferred embodiment, the cells are root cells such as the cells at the root apex.

### Antibodies

The invention also provides monoclonal or polyclonal antibodies to polypeptides of the invention or fragments thereof. Thus, the present invention further provides a process for the production of monoclonal or polyclonal antibodies to polypeptides of the invention.

The term “binds specifically” refers to the ability of the antibody to bind to proteins of the present invention but not other proteins obtained of the plant.

As used herein, the term “epitope” refers to a region of a protein of the invention which is bound by the antibody. An epitope can be administered to an animal to generate antibodies against the epitope, however, antibodies of the present invention preferably specifically bind the epitope region in the context of the entire protein.

If polyclonal antibodies are desired, a selected mammal (e.g., mouse, rabbit, goat, horse, etc.) is immunised with an immunogenic polypeptide (such as SEQ ID NO:24 or SEQ ID NO:26). Serum from the immunised animal is collected and treated according to known procedures. If serum containing polyclonal antibodies contains antibodies to other antigens, the polyclonal antibodies can be purified by immunoaffinity chromatography. Techniques for producing and processing polyclonal antisera are known in the art. In order that such antibodies may be made, the invention also provides polypeptides of the invention or fragments thereof haptenised to another polypeptide for use as immunogens in animals or humans.

Monoclonal antibodies directed against polypeptides of the invention can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced can be screened for various properties; i.e., for isotype and epitope affinity.

An alternative technique involves screening phage display libraries where, for example the phage express scFv fragments on the surface of their coat with a large variety of complementarity determining regions (CDRs). This technique is well known in the art.

For the purposes of this invention, the term “antibody”, unless specified to the contrary, includes fragments of whole antibodies which retain their binding activity for a target antigen. Such fragments include Fv, F(ab′) and F(ab)₂ fragments, as well as single chain antibodies (scFv). Furthermore, the antibodies and fragments thereof may be humanised antibodies, for example as described in EP-A-239400.

Antibodies of the invention may be bound to a solid support and/or packaged into kits in a suitable container along with suitable reagents, controls, instructions and the like.

Preferably, antibodies of the present invention are detectably labeled. Exemplary detectable labels that allow for direct measurement of antibody binding include radiolabels, fluorophores, dyes, magnetic beads, chemiluminescers, colloidal particles, and the like. Examples of labels which permit indirect measurement of binding include enzymes where the substrate may provide for a coloured or fluorescent product. Additional exemplary detectable labels include covalently bound enzymes capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. Further exemplary detectable labels include biotin, which binds with high affinity to avidin or streptavidin; fluorochromes (e.g., phycobiliproteins, phycoerythrin and allophycocyanins; fluorescein and Texas red), which can be used with a fluorescence activated cell sorter; haptens; and the like. Preferably, the detectable label allows for direct measurement in a plate luminometer, e.g., biotin. Such labeled antibodies can be used in techniques known in the art to detect proteins of the invention.

### EXAMPLES

### Example 1

### Materials and Methods

### Al Tolerance Assays

Plants in hydroponic culture were assessed for Al tolerance using a hydroponic solution described as follows, supplemented with a range of AlCl₃ concentrations. The hydroponic solution was made with distilled or deionised water containing 500 μM KNO₃, 500 μM CaCl₂, 500 μM NH₄NO₃, 150 μM MgSO₄, 100 μM KH₂PO₄, 2 μM Fe:EDTA, 11 μM H₃BO₃, 2 μM MnCl₂, 0.35 μM ZnCl₂ and 0.2 μM CuCl₂ adjusted to pH 5.5. The Al screening solution was the same solution as above except it contained 10 μM KH₂PO₄ instead of 100 μM, 2 μM FeCl₃ instead of Fe:EDTA and was adjusted to pH 4.3 instead of 5.5, and was supplemented with AlCl₃ at the stated concentration. The plants were grown using these solutions at a temperature of 18-25° C., usually at 20° C.

Soil experiments used an acid subsoil (10 to 40 cm layer, CaCl₂ extracted pH of 3.9 and 40 μg/g of CaCl₂ extractable Al) derived from Chiltern in Australia (Culvenor, 2004) and a nonallophanic andosol obtained from the Field Science Center at Tohoku University in Japan. In a first experiment, pre-germinated seed, where the longest root was 8 to 35 mm, were planted in 300 g of either un-amended Chiltern soil or Chiltern soil mixed with 0.75 g CaCO₃/100 g soil. Each pot contained a single plant with each combination of genotype and treatment consisting of eight replicates. The experiment was set up as 4 blocks in a green house with 2 replicates of each genotype/treatment placed randomly per block. Pots were weighed daily and watered to 323 g for the acid soil and 316 g for the amended soil. After 4 d growth, seedlings were removed and the longest root re-measured.

In a second experiment, pre-germinated seed, where the longest root was 5 to 26 mm, were planted either in 140 g of moistened acidic andosol (pH 4.5) or in control medium consisting of 80 g of peat moss (Primemix TKS1, Sakata Seed Corporation, Yokohama, Japan; pH 6.5) moistened with 50 mL water and fertilized with nutrients (N: 100-140, P: 80-120, K: 130-190 mg/L). Each pot contained a single plant with each combination of genotype and treatment consisting of six replicates. The experiment was set up as 3 blocks in a growth cabinet with 2 replicates of each genotype/treatment placed randomly per block. The growth cabinet was set at 25° C. and lighting adjusted to 150-200 μmol photons m⁻² sec⁻¹ with a 14-h photoperiod. Pots were weighed daily and watered to 140 g for the acid soil and 130 g for the control medium and roots measured after 4 d growth.

### Real Time Quantitative RT-PCR

Total RNA was prepared (Qiagen RNeasy Plant Mini Kit) from 20 root tips (3 mm long) collected from the various genotypes with 3 biological replicates for each line. The RNA extraction included an on-column DNase step to degrade any contaminating genomic DNA. cDNA was prepared from total RNA (2 μg) as described by Schenk et al. (2000), with the exception that the final elution from the spin column was diluted to 100 μl.

Levels of ALMT1 and control gene expressions were determined by real time quantitative RT-PCR on Rotor-Gene 2000 or 3000 Real Time Cyclers (Corbett Research, Sydney, Australia). One-tenth dilutions were used as template for the quantitative RT-PCR reaction in a total volume of 10 μl as follows: 5 μl SYBR Green JumpStart Taq ReadyMix (Sigma, St. Louis, USA.), 0.5 μl primer mix (50:50 mix of forward and reverse primers at 10 μmol/μl each) and 4.5 μl template. The primers 5′-CGTGAAAGCAGCGGAAAGCC-3′ (SEQ ID NO:8) and 5′-CCCTCGACTCACGGTACTAACAACG-3′ (SEQ ID NO:9) were used for amplification of ALMT1 transcript; 5′-AACAAGACTGCTTTCACCAC-3′ (SEQ ID NO:10) and 5′-TCTCAGAAAGCTCACGGTAG-3′ (SEQ ID NO:11) for amplification of a proton pump transcript from barley (Genbank accession AY136627); 5′-AACAAGACTGCTTTCACCAC-3′ (SEQ ID NO:10) and 5′-TCTCAGAGAGCTCACGGTAG-3′ (SEQ ID NO:12) for amplification of a proton pump transcript from wheat (Genbank accession AY543630); 5′-GAAGGACATCTTCACGGCGATC-3′ (SEQ ID NO:13) and 5′-CACGGCCATGAAGAAGAAGC-3′ (SEQ ID NO:14) for amplification of the wheat phosphate transporter transcript, PT-1 (Genbank accession AF110180); 5′-GAAGGACATCTTCACGGCGATC-3′ (SEQ ID NO:13) and 5′-CACCGCCATGAAGAAGAATC-3′ (SEQ ID NO:15) for amplification of the barley phosphate transporter transcript Pht1-6 (Genbank accession AF543198); and 5′-CTGATCTTCTGTGAAGGGT-3′ (SEQ ID NO:16) and 5′-TGATAGAACTCGTAATGGGC-3′ (SEQ ID NO:17) for amplification of both the wheat and barley 28S ribosomal transcripts (Genbank accession AY049041). Cycling conditions were as follows: 5 min at 94° C. followed by 45 cycles of 15 s at 94° C., 15 s at 55° C., and 20 s at 72° C. At the end of the cycling the samples were incubated at 40°C. for 5 min then at 55° C. for 1 min followed by a melting curve program (55° to 99° C. in one degree increments with a 5 s hold at each temperature).

### Barley Transformation

The method used for the transformation of barley was based on the method of Tingay et al. (1997). The gene constructs in binary vectors were introduced into a highly virulent Agrobacterium strain (AGL) by tri-parental conjugation, which was then used to introduce the T-DNA containing the transgene and the selectable marker gene (encoding hygromycin resistance, expressed from the CaMV35S promoter) into regenerable cells of the scutellum of immature barley embryos, as follows.

Developing barley seeds from the variety Golden Promise, 12-15 days after anthesis, were removed from the growing spike of greenhouse grown plants, and sterilised for ten minutes in 20% (v/v) bleach followed by rinsing once with 95% ethanol and seven times with sterile water. Embryos (approx 1.5 to 2.5 mm in size) were then removed from the seeds under aseptic conditions and the axis cut from each embryo. The embryos were placed cut side down on a petri dish containing callus induction medium. The Agrobacterium transconjugants were grown in MG/L broth (containing 5 g mannitol, 1 g L-glutamic acid, 0.2 g KH₂PO₄, 0.1 g NaCl, 0.1 g MgSO₄.7H₂O, 5 g tryptone, 2.5 g yeast extract and 1 μg biotin per litre, pH 7.0) containing spectinomycin (50 mg/l) and rifampicin (20 mg/l) with aeration at 28° C., to a concentration of approximately 2-3×10⁸ cells/ml, and then approx 300 μl of the cell suspension was added to the embryos in a petri dish. After 2 min, excess liquid was tipped from the plate and the embryos were flipped so that the cut side (axil side of the scutellum) was upwards. The embryos were then transferred to a fresh plate of callus inducing medium and placed in the dark for 2-3 days at 24° C. The embryos were transferred to callus inducing medium with selection (50 μg/ml hygromycin and 150 μg/ml timentin).

Embryos remain on this media for 2 weeks in the dark at 24° C. Healthy callus was then divided and placed on fresh selection media and incubated for a further two weeks at 24° C. in the dark. Following this, the embryos were incubated at 24° C. in the light for 2 weeks on regeneration medium containing cytokinin and transferred to rooting media containing cytokinin and auxin for three 2 week periods. Juvenile plants were then transferred to soil mixture and kept on a misting bench for two weeks and finally transferred to a glasshouse.

### Example 2

### Expression of Aluminium Tolerance Gene in Transgenic Barley

### Gene Constructs

The coding region of the ALMT1-1 gene (referred to as ALMT1, nucleotide sequence: GenBank accession AB081803) was amplified by reverse-transcription PCR (RT-PCR) from polyA⁺ RNA as described by Sasaki et al. (2004). The resulting amplified fragment was digested with SalI and NotI and inserted into the plasmid pTH2 (Chiu et al., 1996) by replacing the GFP sequence to yield pTH-ALMT1-1. After the ALMT1 coding region was verified by sequencing, plasmid pTH-ALMT1-1 was digested with SalI and NotI, the ALMT1-1 fragment blunted by end-filling and then inserted into the Sma site of pWUbi (Wang et al., 1998). The orientation of the insert with respect to the ubiquitin promoter such that the sense strand would be expresssed was verified. Then, pWUbi was digested with NotI to excise the fragment containing the expression cassette containing the ubiquitin promoter, the ALMT1 coding region and the terminator. This fragment was then inserted into the NotI site of the binary vector pWBVec8 (Wang et al., 1998), the orientation of the insert with respect to the selectable marker was determined and the plasmid was introduced into Agrobacterium by triparental mating. Southern blots were performed using standard procedures (Sasaki et al., 2004).

The resultant plasmid, pWBVec8-Ubi-ALMT1 was a binary vector having a T-DNA containing the ALMT1 coding region under the control of the ubiquitin promoter for expression in the roots (and elsewhere in the plants), and was used for Agrobacterium-mediated transformation of barley.

### Plant Materials and Plant Transformation

The barley cultivar Golden Promise was transformed using Agrobacterium tumifaciens containing the transgene(s) as described in Example 1. Control plants were generated by transformation with the binary vector pWBVec8 lacking the gene insert and from wild-type plants that were derived from plants that had progressed through tissue culture (regenerated) but were not transformed.

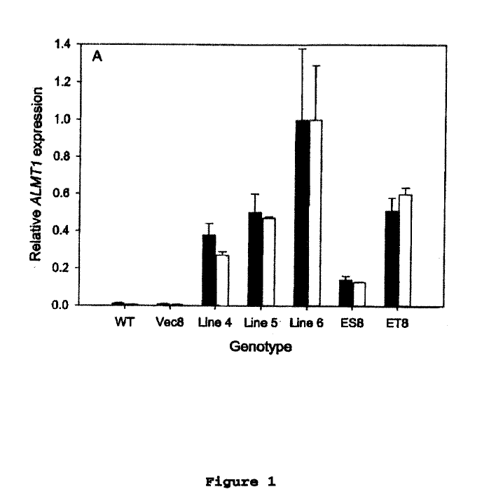

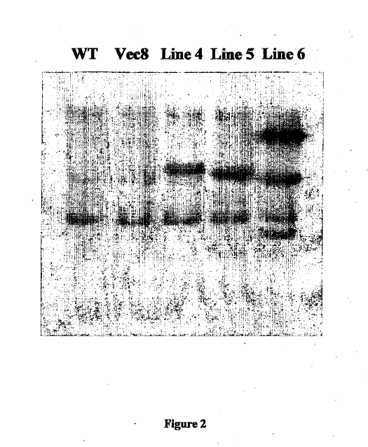

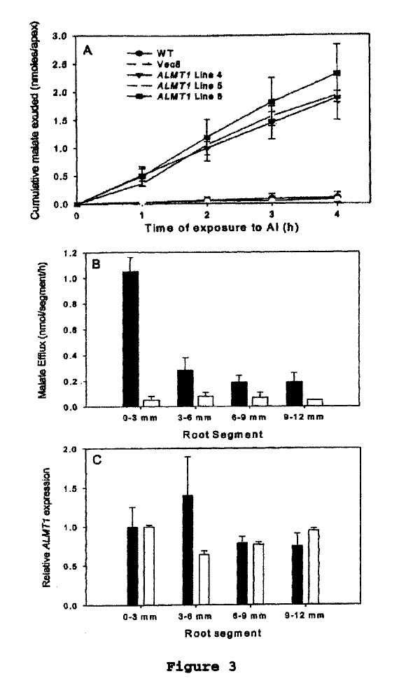

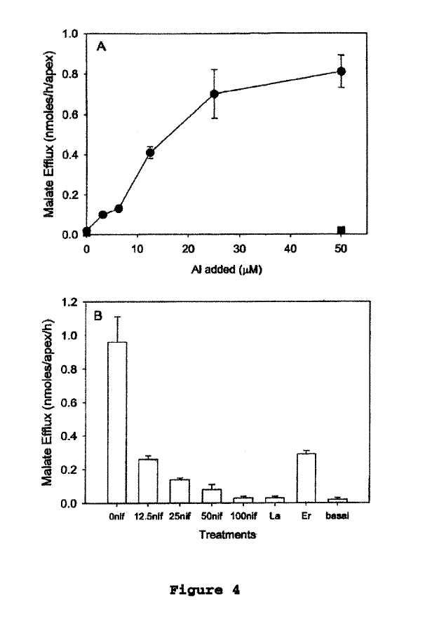

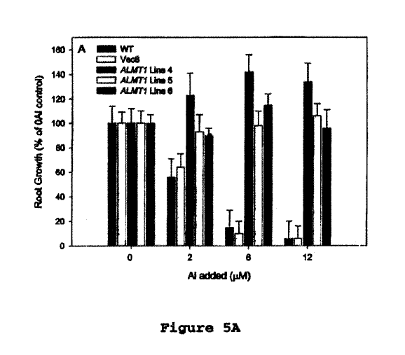

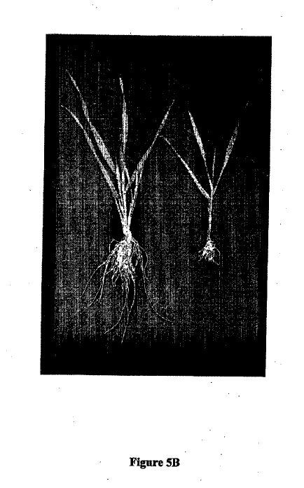

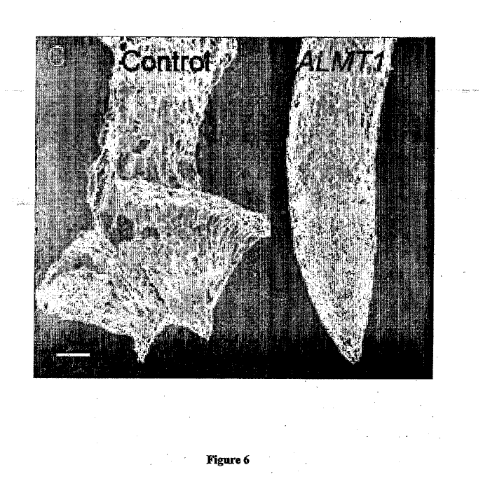

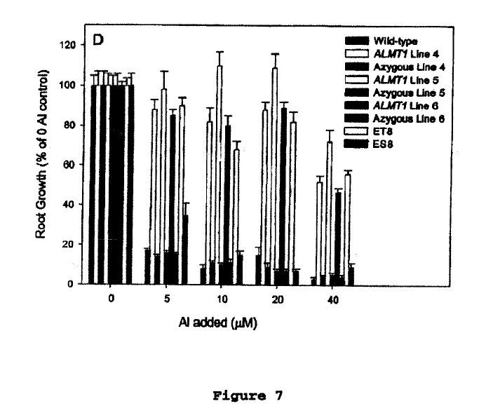

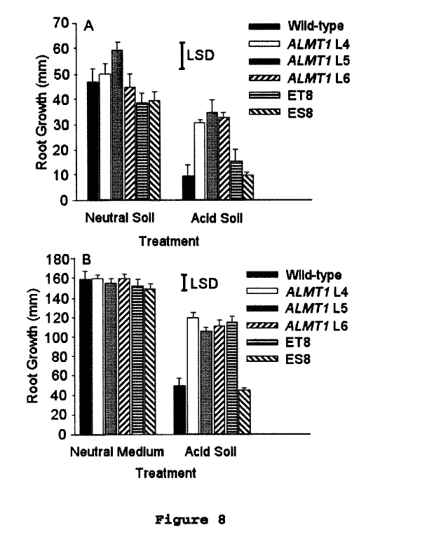

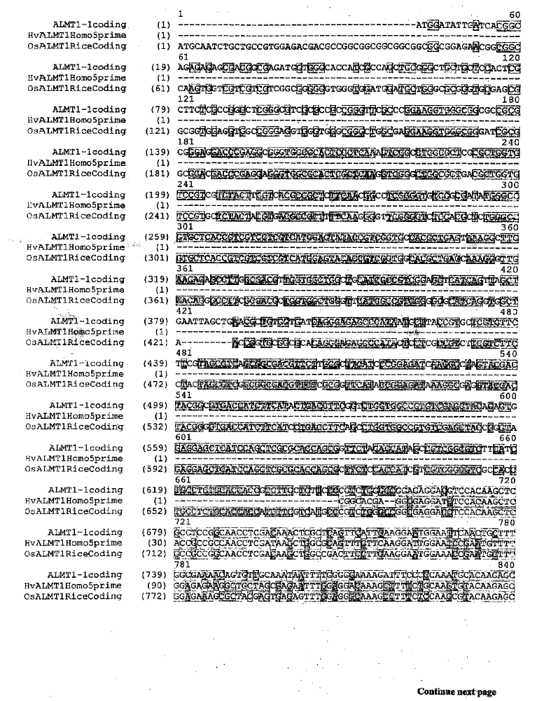

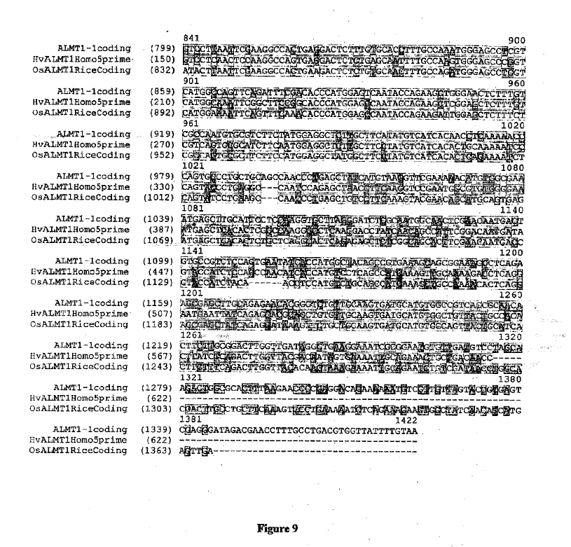

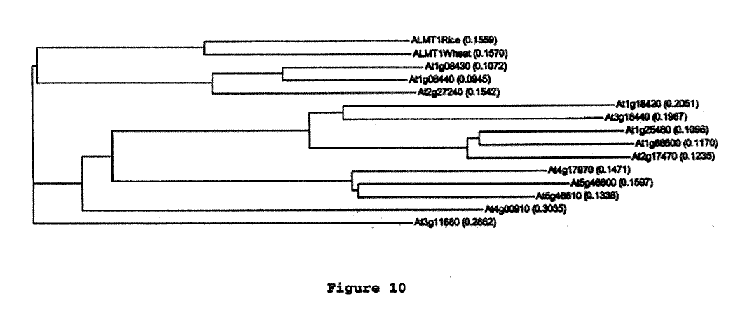

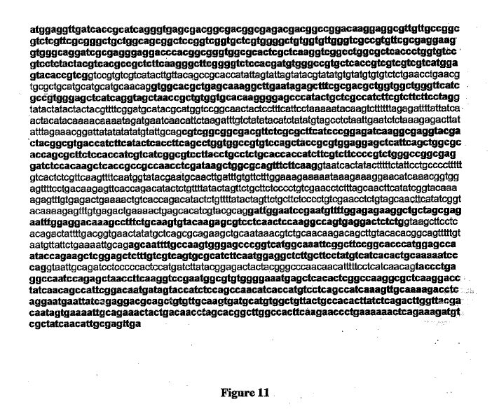

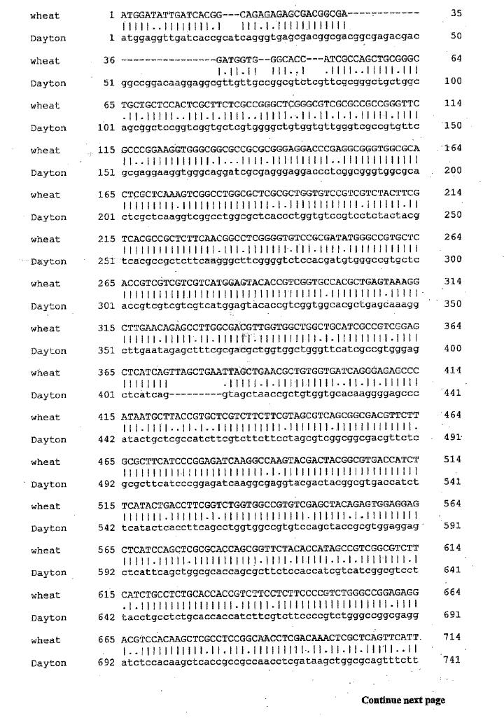

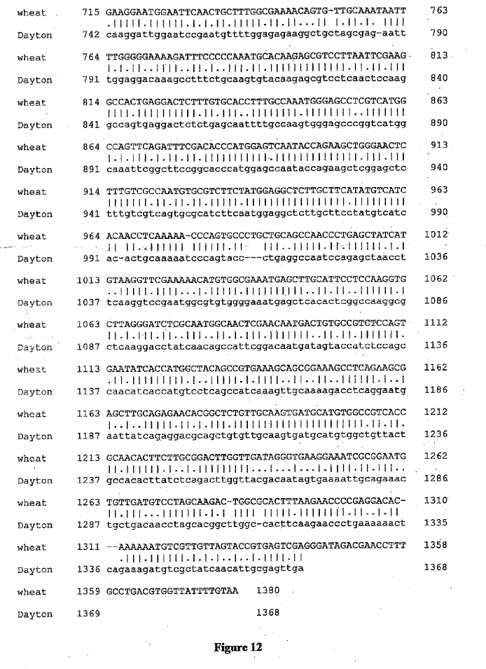

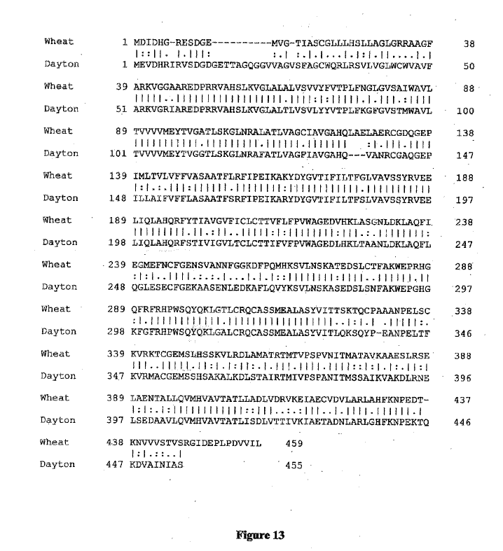

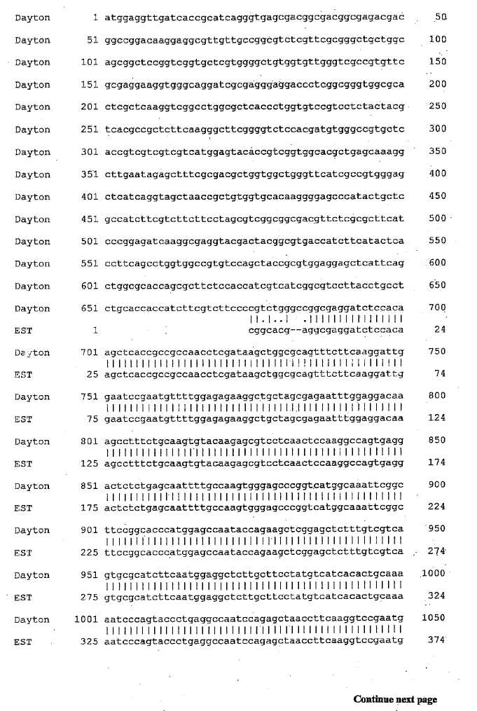

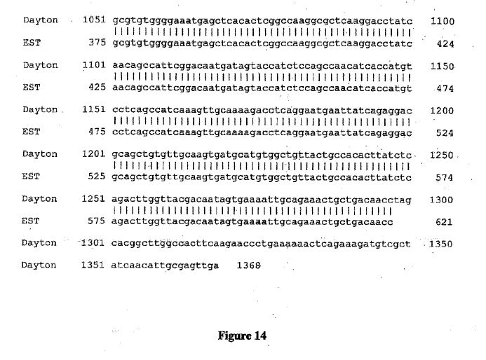

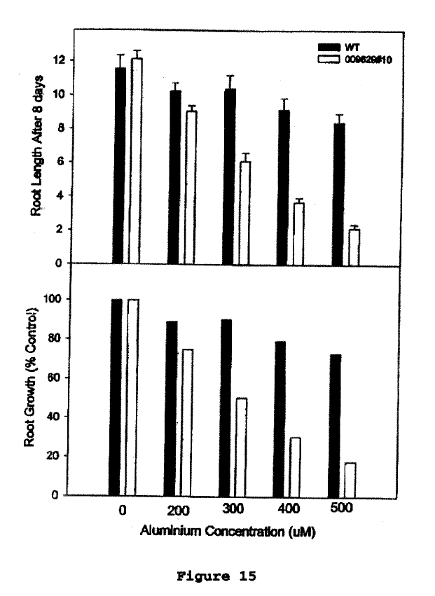

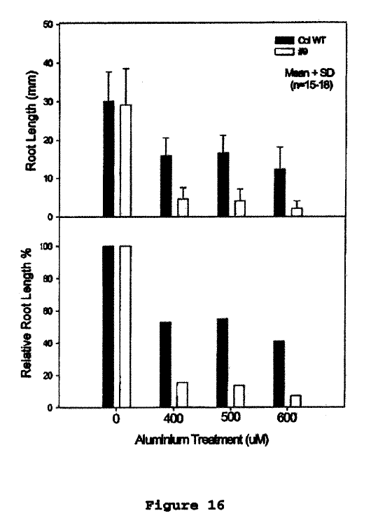

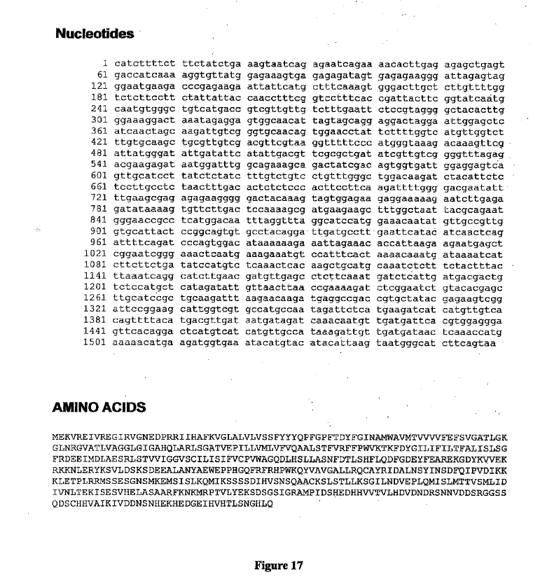

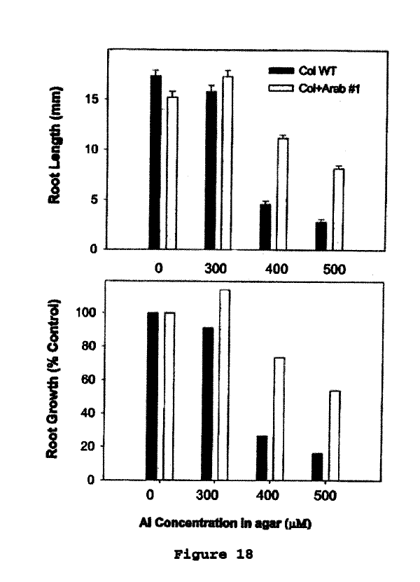

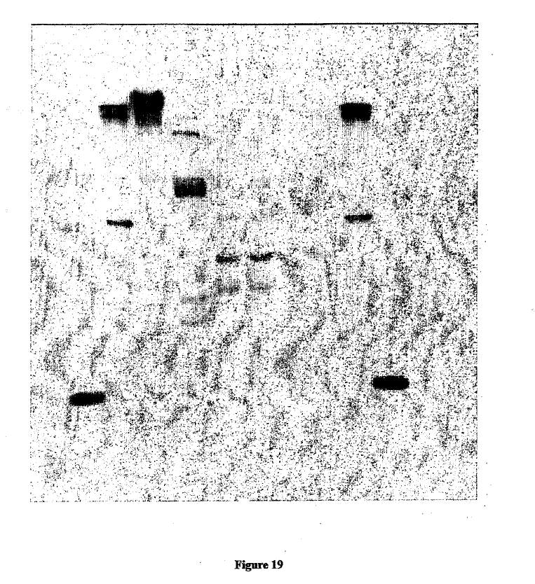

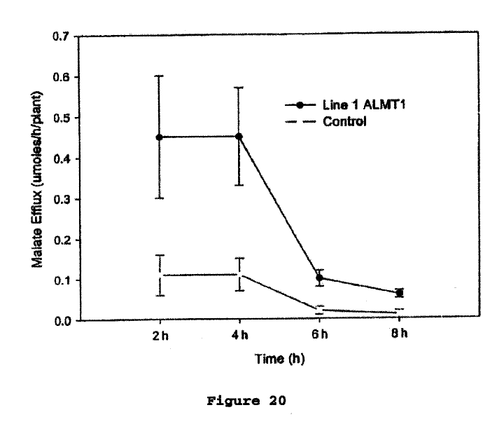

Agrobacterium tumefaciens transformation was used to introduce the ALMT1 gene under the control of the ubiquitin promoter into barley. Twenty-five primary transformants that were hygromycin resistant were obtained and shown to contain the ALMT1 gene by PCR or Southern blot hybridization. These were screened for expression levels of the ALMT1 gene by real time quantitative reverse transcriptase-mediated PCR (RT-PCR) using the method described in Example 1. Analysis of root apices by RT-PCR verified ALMT1 expression in these lines and showed that the expression levels were similar to an Al-tolerant wheat when either a phosphate transporter or proton pump gene was used to normalize expression (FIG. 1). Southern blots showed the presence of single (lines 4 and 5) or multiple insertions (line 6) of the gene while a band common to all genotypes including control plants (Golden Promise) indicated the presence of a related gene in the barley genome (FIG. 2).

### Example 3

### Transgenic Barley Expressing ALMT1 Show an Al-Activated Malate Efflux

The three highest expressing transformed barley lines, designated lines 4, 5 and 6, were selected for detailed analysis as follows.

Primary transformants (T0) were maintained as clonal populations by taking tillers and growing them separately. In this way up to 20 individual plants were maintained for each transgenic line, allowed a homogenous population of plants to be tested for each line. The clonal plants were maintained by hydroponic culture in the Al screening solution as described above. Several T0 plants of each line were transferred to soil and grown to maturity to produce seed of the T1 generation. Seed collected from T1 plants (T2 generation) were germinated and grown in hydroponic culture using a modified Al screening solution (which was the same as described above except that it contained 10 μM KH₂PO₄, 2 μM FeCl₃ instead of Fe:EDTA and was adjusted to pH 4.3) with AlCl₃ added to 10 μM. In this way lines homozygous for ALMT1 and sister lines azygous for ALMT1 (“null segregants”) were identified and confirmed independently by either their level of hygromycin resistance or by PCR to amplify the ALMT1 gene.

### Malate Efflux

Malate efflux from root segments was assayed using a modification of methods described by Ryan et al. (1995a). Unless stated otherwise, 4 root apices 3 mm long were incubated with shaking in 1 ml of 0.2 mM CaCl₂ (pH 4.3) for approximately 1 h. The apices were then rinsed with the CaCl₂ solution three times and replaced with 1 ml of the same solution (control) or 1 ml of treatment solution (0.2 mM CaCl₂ with added treatment at pH 4.3). After an incubation that typically lasted for 1 h, the solution was removed, dried and the residue re-suspended in 200 μl of buffer used for the spectrographic assay of malate (0.25 M glycine, 0.20 M hydrazine and 2.7 mM NAD adjusted to pH 9). A subsample (1001) was transferred to a micro-cuvette and the increase in absorbance at 340 nm measured 6 min after addition of 1 μl of malate dehydrogenase (5 mg/ml) was used to calculate the amount of malate by comparison to a standard curve.

Plants of all three ALMT1 lines that were tested showed an Al-activated malate efflux from root apices that was absent from either wild-type plants or plants transformed with the vector alone (FIG. 3A). Efflux was maintained from the excised root apices over at least 4 h of Al exposure.

The root apex (approximately 3 mm at the root tip) represents the most Al-sensitive part of the root (Ryan et al., 1993) and is the region that specifically possesses the Al-activated efflux of malate in Al-tolerant wheat (Ryan at al., 1995a). The extent of malate efflux from various segments along the roots of the transgenic barley was therefore determined. Since ALMT1 expression in the transgenic barley was under the control of the ubiquitin promoter which confers a high level of constitutive expression throughout the plant, the level of Al-activated malate efflux was also determined for more mature root segments. The results (FIG. 3B) showed that while older root segments also showed an Al-activated efflux of malate, it was considerably less than that observed from the root apex. The degree of transgene expression in root segments along the root was also determined and correlated with the malate efflux to establish whether the level of transgene expression was the cause of this. To do this, the level of gene expression of the transgene ALMT was normalised to that of two other transporter genes by real-time RT-PCR, as described in Example 1. The data (FIG. 3C) for transgenic line 5 showed that ALMT1 was expressed at similar levels in all root segments analyzed, when normalized to the expression of the two transporter genes (FIG. 3C) or a ribosomal RNA. This indicated that the level of gene expression was not the limiting factor in determining the extent of malate efflux.

Malate efflux from roots of line 5, a transgenic line with a single ALMT1 insert, was characterized in greater detail. The efflux responded to Al concentration in a dose-responsive manner, increasing to about 25 μM added Al (FIG. 4A) and then starting to level off. Malate efflux was inhibited by the anion channel blocker niflumic acid. Lanthanum (La) was ineffective in activating malate efflux and erbium (Er), a rare earth element, was capable of eliciting malate efflux, albeit at a lower level than observed for Al (FIG. 4B). This was consistent with the results of Kataoka et al. (2001) who found that Er along with a range of other rare earth elements was capable of eliciting malate efflux from Al tolerant wheat.

Al-activated malate efflux from root apices of transgenic barley expressing ALMT1 was accompanied by the efflux of K⁺. Net K⁺ effluxes from excised apices of line 5 were calculated by subtracting corresponding values obtained for the vector only control line resulting in an efflux of 0.4±0.3 nmoles K⁺/h/apex in the absence of Al and an efflux of 5.2±1.0 nmoles K⁺/h/apex in the presence of 50 μM Al (means±SE; n=4).

### Example 4

### Expression of ALMT1 in Barley Confers Enhanced Al Tolerance

### Results

Al tolerance was assessed by determining root elongation of plants grown in hydroponic culture in the continual presence of added Al. The three barley T0 lines that were transformed with ALMT1 and expressing the transgene in the roots were tested in a range of Al levels. All three showed robust root growth in hydroponic culture at Al concentrations that severely inhibited roots of control plants (FIGS. 5A and 5B). Whereas control plants showed a 50% inhibition of root growth at 2 μM AlCl₃ under the conditions used, and 85-90% inhibition at 6 μM, the transgenic plants did not exhibit any significant inhibition of root growth at these levels or at 12 μM. Line 5 did not show significant inhibition of root growth even at 20 μM AlCl₃. The ALMT1 gene therefore provided a dramatic enhancement of Al tolerance on the barley plants, to a level that has not been seen previously.

When examined under the microscope, root apices of the ALMT1 transformed plants were unaffected by the Al whereas those of the controls were severely damaged and malformed (FIG. 6).

Progeny of the T0 plants were also examined. Homozygous T2 lines (second generation from the T0) expressing ALMT1 were Al tolerant in hydroponic culture compared to azygous sister lines or the wild type parental line (FIG. 7). Azygous sister lines derived from the same transformation events that generated the ALMT1-expressing lines provided ideal controls as they are plants that have experienced the same tissue culture conditions during the transformation procedure. The level of tolerance in hydroponic culture was comparable to ET8, the Al tolerant wheat line which was the original source of the ALMT1 gene.

Staining of roots previously exposed to Al solutions with hematoxylin is another method commonly used to assess Al tolerance in cereals (Polle et al., 1978; Echart et al., 2002). Hematoxylin forms a purple-red complex with Al and staining with this compound therefore provides an indirect measure of non-complexed Al in root apices, with the intensity of staining being correlated with sensitivity to Al in wheat. Root apices of control plants became stained with hematoxylin suggesting that they had accumulated Al while those of the ALMT1 transgenics remained clear. Some swelling was also apparent behind the tip of the control line which is a typical early symptom of Al toxicity.

To determine whether the Al tolerance of the transgenic barley plants expressing ALMT1 was apparent in soil, homozygous plants of the three lines were grown in a severely acidic soil that contained high concentrations of soluble Al. ALMT1-expressing transgenics clearly showed better root growth in the acid soil than their azygous sister lines or wild type controls (FIG. 8A). When the soil was neutralized with CaCO₃, root growth of all barley lines was similar regardless of genotype. The toxicity of the soil was apparent on the ET8/ES8 wheat lines where root growth of the Al-tolerant ET8 line was inhibited on the un-amended soil to such an extent that it did not differ significantly from the sensitive ES8 line. When the lines were grown on a less severely acidic soil, the homozygous lines expressing ALMT1 showed better root growth than wild-type barley and ET8 wheat grew significantly better than ES8 (FIG. 8B).

### Discussion

The barley cultivar used in this current study (cv Golden Promise) was very susceptible to Al toxicity and did not possess an Al-activated efflux of malate. When expressing an ALMT1 gene in the roots, the transgenic barley plants exhibited an Al-activated malate efflux which was associated with an Al tolerance phenotype. The latter was shown using a hydroponic growth system and by growth in acid soils. The tolerance was shown at Al levels (up to at least 20 μM AlCl₃) that were highly toxic to root growth in the control, untransformed plants. These data provide the first evidence that ALMT1 is capable of conferring Al tolerance to intact transgenic plants and further support the notion that ALMT1 is a major gene for Al tolerance in wheat. In contrast, rice (Sasaki, et al., 2004) and tobacco transformed with an ALMT1 gene construct did not exhibit increased Al tolerance.

In addition, the data provide strong evidence in support of the model of malate efflux as a tolerance mechanism in barley as well as wheat. According to this model, secreted malate binds Al into a non-toxic form and protects the root apex from damage.

Similar to Al-tolerant wheat genotypes (Ryan et al., 1995a; Kataoka et al., 2001), malate efflux in the transgenic barley showed an Al dose response, was inhibited by the anion channel-blocker niflumic acid, was activated by Er and was accompanied by K⁺ efflux. These features are consistent with ALMT1 encoding an Al-gated (activated) anion channel that is specifically permeable to malate. The rate of malate efflux from root apices of the transgenic barley (approx. 1 nmole/apex/h) was similar to that found in Al-tolerant wheat genotypes (Ryan et al., 1995a; Ryan et al., 1995b). Consequently, the levels of Al tolerance in hydroponic culture of tolerant wheat and the transformed barley were similar. When grown in a severely acidic soil (FIG. 8A), however, the transformed barley showed better root growth than Al tolerant wheat.

The finding that a single gene was able to confer an Al-activated malate efflux in barley to a similar level found in Al-tolerant genotypes of wheat suggested that the biosynthesis of malate was not a rate limiting step for its efflux from root apices of barley as concluded previously for Al-sensitive wheat. While there is evidence that over-expressing genes involved in organic acid biosynthesis can increase the content of organic acid anions and their subsequent efflux from, roots of some species, it appears that transport of organic acid anions across the plasma membrane is the major factor that limits efflux in species such as barley and wheat.

The ALMT1 gene was expressed under the control of the ubiquitin promoter which has been shown to confer high level constitutive expression of transgenes in both meristimatic and mature regions of roots (Schunmann et al., 2003). By contrast, expression of ALMT1 in wheat under the control of its native promoter is restricted to the apical 2 to 3 mm of the root which coincides with the region of malate efflux. However, despite using a constitutive promoter to express ALMT1, the observed malate efflux was primarily from the root apex and in that aspect was similar to the phenotype seen in Al-tolerant wheat. This may help avoid the potential metabolic costs to the plant associated with a high level of malate efflux from all root tissues.

These data show the use of gene transformation technology to alter a normally sensitive crop species, in this case barley, so that it can be grown effectively in acid soils.

### Example 5

### Identification of Other Al Tolerance Genes

To identify other Al tolerance genes, the ALMT1 protein sequence was used to search sequence databases for homologous proteins. A search of the protein database (National Center for Biotechnology Information, http://www.ncbi.nlm.nih.gov/) identified a rice gene (accession number CAD40928) that encoded a protein of unknown function with 69% amino acid sequence identity to ALMT1 and a barley EST (Accession No. BU993212). A comparison of the wheat ALMT1 gene, the barley EST and the rice gene (cDNA) sequences is shown in FIG. 9. Additional putative proteins encoded by ESTs from rice and Arabidopsis thaliana showed 30-42% identity to ALMT1 (e.g. accession numbers: AAF02135, AAD42005 and AAL86482). A phylogenetic relationship based on sequence similarity among ALMT1 and the Arabidopsis homologues is shown in FIG. 10.

A full-length ALMT1 homolog from barley was cloned as follows. A bacterial artificial chromosomal (BAC) library of the barley cultivar Morex (aluminium sensitive) was hybridised with a ³²P labeled probe made from the coding region of the wheat ALMT1-1 gene. The hybridization solution comprised 6×SSC, 50 mM Tris-Cl, mM EDTA, 5×Denhardts solution, 0.2% SDS and 10% dextran sulphate (details for SSC and Denhardts solution given in Sambrook et al. 1989, supra). Filters were hybridized at 65° C. for 16 h and then washed three times for 20 min each with a solution comprising 2×SSC and 0.1% SDS at 65° C. From the screening, a colony that hybridized strongly was identified and the BAC clone purified. To subclone the barley DNA, the BAC clone was digested with HindIII and fragments inserted into the HindIII site of pBluescript (Statagene). The resulting library of subclones derived from the BAC clone was screened for colonies that contained ALMT1-like sequences using the same coding region probe. Plasmids from positive colonies were isolated and sequenced to yield a genomic sequence for a barley ortholog of the wheat ALMT1 gene which was named HvALMT1 (FIG. 11) (SEQ ID NO:22).

Based on the predicted start and stop codons and the introns in relation to the wheat ALMT1 gene, primers were designed that amplified the coding region of HvALMT1 using reverse transcription PCR from root mRNA of the cultivars Morex (aluminum sensitive) and Dayton (aluminum tolerant). The forward primer was ATGGAGGTTGATCACCGCATC (SEQ ID NO:27) and reverse primer was TCAACTCGCAATGTTGATAGCG (SEQ ID NO:28). The HvALMT1 coding regions of Morex and Dayton were sequenced and found to possess several single nucleotide polymorphisms. More specifically, at position 186 Dayton has a T whereas Morex has an A and at position 1288 Dayton has a G where as Morex has an A (numbering as provided in FIG. 12). Only one of these differences results in an amino acid difference with Dayton having an Ala whereas Morex has a Thr at position 430 of the protein.

FIG. 12 provides, an alignment of the wheat ALMT1 coding sequence and the barley cv Dayton HvALMT1 coding sequence which are 74.9% identical. FIG. 13 provides an alignment of the corresponding proteins which are 66.9% identical.

Provided in FIG. 14 is an alignment of EST Accession No. BU993212 (SEQ ID NO:5) and a cDNA sequence encoding barley Dayton cv HvALMT1. As can be seen from FIG. 14, the first 674 nucleotides and last 71 nucleotides of the open reading frame are missing from the EST.

To over-express the barley ALMT1 homolog in barley, the amplified full length cDNA obtained from cultivar Dayton using the above primers was inserted into pGemT-easy, transformed into E. coli and plasmids from several colonies purified and sequenced. One plasmid which yielded an identical sequence to that predicted by the genomic sequence was used to prepare constructs for transformation of barley. The coding region of HvALMT1 was digested out of the pGemT-easy clone with EcoRI and ligated into the EcoRI site of the vector pWUbi, containing the constitutive ubiquitin promoter, to generate pHvALMT1:WUbi. The resulting plasmid was sequenced to verify the correct orientation of the coding region relative to the promoter-intron structure in the vector. pHvALMfT1:WUbi was digested with NotI and the fragment that contained the promoter:intron:HvALMT:terminator expression cassette was ligated into the NotI site of pVec8. The resulting plasmid (pHvALMT1:Vec8) was introduced into Agrobacterium by triparental mating and used to transform barley as described in Example 1. Up to 30 plants were generated which, based on their resistance to hygromycin, were determined to be successfully transformed. These transformants will be analysed for expression of the introduced HvALMT1gene by RT-PCR or Northern blot hybridisation, the level of organic acid efflux (malate and citrate) using the method described in Example 3 and for the level of Al tolerance using the method described in Example 1. It is predicted that the transformants expressing the highest levels of the introduced HvALMT1 gene will show the highest level of organic acid efflux from their roots and improved aluminium tolerance.

The wheat ALMT1 gene and the homologues in rice and Arabidopsis possess a conserved domain of the UPF0005 protein family, previously uncharacterized (http://www.ncbi.nlm.nih.gov). The data presented herein show that wheat ALMT1 encodes an Al-activated malate transporter.

### Example 6

### Isolation of a Gene from Arabidopsis and Demonstration of its Role in Al Tolerance

An Arabidopsis line with an insertional mutation in one of the ALMT1 homologues was obtained from the Arabidopsis Biological Resource Center (ABRC; Ohio State University 309 Botany and Zoology Bldg, Columbus Ohio 43210, USA, in collaboration with the Salk Genomic Analysis Research Laboratory http://signal.salk.edu/tabout.html). The line obtained from ABRC was designated as SALK_{—}009629 and has an insertional mutation in the At1g08430 gene. The ecotype used to produce the insertional mutations was Columbia-0 (CS60000) which corresponds to the sequenced Arabidopsis genome. Ten T1 seeds of SALK_{—}009629 were germinated and grown on soil. After 4 weeks, approximately 100 mg of leaf tissue was collected from each plant and stored at −80° C. Plants were then grown to maturity and seed collected from each and stored separately. DNA was extracted from the leaf tissue using the method of Edwards et al. (1991).

Plants that were homozygous for the insertion were identified by PCR as follows. Principles of this PCR test and primer design are described on a number of pages on the Web site for the Salk Institute Genomic Analysis Research Laboratory (http://signal.salk.edu/tdnaprimers.html). Three primers were required for this PCR test as follows: the Left Border primer (LB) which recognized the sequence of the insert (5′-GCGTGGACCGCTTGCTGCAACT-3′) (SEQ ID NO:18), and two gene specific primers that recognize the sequence flanking the insert: the Left Primer (LP; 5′-GAAGCTGCAAGAAAAAGTTTAGTTA-3′) (SEQ ID NO:19) and the Right Primer (RP; 5′-CCACTGTTGCACCCGACAATC-3′) (SEQ ID NO:20). Primers were chosen such that a PCR reaction using DNA from plants homozygous for the insertion produced a single band (approx. 400 bases), plants heterozygous for the insert produced two bands (approx. 400 bases and 900 bases) and the null segregant (wild-type) produced a single band (approx 900 bases). Each PCR mix (20 μl) included: 10 μl QIAGEN Master Mix™ (2×), 2 μl DNA template, 1 l Primer LB (10 μmol μL⁻¹), 1 l Primer LP (10 μmol μL⁻¹), 1 l Primer RP (10 μmol μL⁻¹), 5 l H₂O. The PCR cycling conditions were: 95° C.-15 min, 30 cycles of (95° C., 30 s; 52° C., 30 s; 72° C., 60 s); 72° C., 2 min. Using this procedure two T2 plants homozygous for the insertional mutation were identified. Seeds of these plants were designated Lines #9 and #10.

### Root Growth Assays

A root growth assay was used to compare the Al tolerance in wild-type and mutant Arabidopsis plants as follows. Root growth solution (RGS) was made containing 625 μM KNO₃, 250 μM CaCl₂, 250 μM MgSO₄, 250 μM KH₂PO₄, 6.25 μM Na₂EDTA, 6.25 μM FeCl₃, and 0.5 ml of micronutrient solution per liter. The micronutrient solution contained 1.425 g H₃BO₃, 0.895 g MnCl₂.4H₂O, 0.2 g ZnSO₄.7H₂O and 0.066 g CuCl₂.2H₂O per liter. The RGS was adjusted to pH 6.0 with NaOH. A sterile agar media containing nutrients and a range of AlCl₃ concentrations was prepared as follows:

| | |
|---|---|
| 220 ml | RGS |
| 0.396 g | succinic acid |
| 4.95 g | agar (BBL ™) |

made up to 660 ml and pH to 4.8 with 1N NaOH, then autoclaved to sterilise. Once the media had cooled to about 40-50° C., 60 ml was poured into a sterile beaker, mixed with a volume of sterile AlCl₃ and poured into a sterile petri dish. The plates were left to set solid in a sterile hood. The 25 mM AlCl₃ stock solution was prepared and filter-sterilised. The following amounts were added to 60 ml of nutrient media for each plate:

| ml stock for a final conc | μM Al on agar plate |
|---|---|
| 0 ml stock for a final conc | 0 μM Al on agar plate |
| 0.48 ml stock for a final conc | 200 μM Al on agar plate |
| 0.72 ml stock for a final conc | 300 μM Al on agar plate |
| 0.96 ml stock for a final conc | 400 μM Al on agar plate |
| 1.2 ml stock for a final conc | 500 μM Al on agar plate |
| 1.44 ml stock for a final conc | 600 μM Al on agar plate |

Seed of wild-type Arabidopsis and homozygous mutant lines were surface-serilised with chlorine gas as described by Delhaize et al. (2001). About 20 seeds from each line were placed along a mid-line on each agar test plate. After 8-14 d the lengths of the two longest roots on each seedling were measured with a ruler.

### Results

Inhibition of root growth is the most common symptom of aluminium toxicity in plants. The amount of root growth of wild-type Arabidopsis (ecotype Columbia-0) and mutant lines were compared after 14 days growth on agar plates containing toxic concentrations of AlCl₃ at 200-600 μM. The total root length and relative root growth for the mutant and wild-type genotypes are shown in FIGS. 15 and 16. The root growth of the mutants plants was significantly slower than the wild-type plants. These data show that the insertion of an inactivating element in the At1g08430 gene of Arabidopsis caused an increasing sensitivity to aluminium and proved that this gene has a role in providing aluminium tolerance.

### Example 7

### Transformation of Plants with the At1g08430 Gene

The nucleotide and amino acid sequences of the At1g08430 gene are shown in FIG. 17. This gene encodes a protein with approximately 40% amino acid sequence identity to the ALMT1 protein of wheat. To confirm the activity of this protein when overexpressed in Arabidopsis, the coding region of Atg08430 (AtALMT1) was inserted into the vector pART7 and the resulting promoter-AtALMT1-terminator fragment excised with NotI. This fragment was then inserted into the NotI site of the binary vector pPLEX502 to yield pAtALMT1:PLEX502. pAtALMT1:PLEX502 was introduced into Agrobacterium by triparental mating and the transconjugants used in transformation experiments to produce plants of Arabidopsis ecotypes Columbia and Landsberg containing the gene. Expression of AtALMT1 was confirmed in several selected transgenic lines for each ecotype. Typical data (FIG. 18) showed enhanced Al tolerance based on root growth of the transgenic lines.

The open reading frame encoding the At1g08430 gene may be inserted into the binary vector pWBVec8 as described above for the ALMT1 wheat gene and transgenic barley may be produced by the method as described in Example 2. The transformed barley plants may be tested for Al tolerance and lines with improved tolerance thereby identified.

### Example 8

### Identification of Further Gene Homologs

Genomic DNA was extracted from Arabidopsis, two wheat genotypes ET8 and ES8, two varieties of subterranean clover (Trifolium subterreaneum) as well as Brassica napus and Brassica juncea using the following procedure. Shoot tissue was homogenized in a mortar and pestle with 5 ml of extraction buffer (0.1 M Tris-HCl pH 8.2, 0.125 M EDTA, 0.2 M NaCl, 40 μg ml⁻¹ RNase A, 0.5% (w/v) SDS). After shaking the mixture at 37° C. for 1 to 2 h, 0.5 mg proteinase K was added and the mixture incubated for a further 2 h at 50° C. The sample was centrifuged at 6000 g for min, and the supernatant solution was placed on ice. The DNA was precipitated with ethanol, retrieved by spooling, rinsed in ethanol and then dissolved in TE. The DNA extracted with phenol:chloroform:isoamylalcohol (25:24:1) and then re-precipitated with ethanol before being dissolved in water. 15 μg DNA from each extraction were digested with 3 uL BamH1 in a total volume of 30 μL overnight at 37° C. The digested DNA was separated on a 1% agarose gel using TAE buffer. The blot was denatured and fixed to a Hybond N⁺ membrane as described by Sambrook et al. (1989, supra).

A probe was prepared from the coding region of the Arabidopsis gene At1g08430. Forward and reverse primers were used to amplify a 440 bp fragment using forward (5′-ATGGAGAAAGTGAGAGAGATAG-3′) (SEQ ID NO:21) and reverse (5′-CCACTGTTGCACCCGACAATC-3′) (SEQ ID NO:20) primers using the HotStarTaq™ Master Mix Kit (Qiagen) according to the manufacturers instructions. The amplification used 35 cycles of 94° C., 30 s; 60° C. anneal, 30 s; 72° C., 60 s in the presence of radio-labelled nucleotide triphosphate. The PCR reaction products were gel purified on a 1% agarose gel with modified TAE buffer. PCR products were cut from, the gel and purified with Amicon Ultrafree-DA™ spin columns.

Prehybridisation solution was prepared as follows: 5.0 ml dH₂O, 3.0 ml 20×SSC, 0.5 ml 1M Tris (pH 8), 0.2 ml 0.5M EDTA, 1.0 ml 50×Denhardts solution, 0.1 ml 20% SDS. Denatured sonicated salmon sperm DNA was added at 500 μg/10 mLs. The hybridisation solution used was the same as the prehybridisation except 2 mL 50% dextran sulfate was added and only 3 mL dH₂O, in addition to the probe. Prehybridisation (6 h) and hybridisation (24 h) occurred at 65° C. The filters were then washed three times for 15 min at 65° C. in 2×SSC+0.1% SDS and then once for 15 min in 1×SSC+0.1% SDS. These conditions correspond to high stringency hybridization conditions. The blot was then exposed to Kodak film.

Southern blot hybridisation analysis was used to determine whether homologues of the Arabidopsis At1g08430 gene were present in other plant species, particularly other dicotyledonous species. The results (FIG. 19) showed that a probe prepared from the 5′ end of At1g08430 hybridised under high-stringency conditions with sequences from Brassica napus, Brassicajuncea and Trifolium subterreaneaum as well as wheat. This demonstrated that gene homologs were present in other plant species. Such homologs are isolated by the preparation of gene libraries from these species in a phage or plasmid vector and hybridization with a probe such as the one described above. The isolated genes are then characterized by standard methods.

### Example 9

### Transformation of Tobacco with the Wheat ALMT1 Gene

Sasaki et al. (2004) reported that rice plants transformed with the wheat ALMT1 gene did not exhibit increased Al tolerance even though expression of the transgene was associated with an Al-activated efflux of malate from the roots. They also reported that expression of the ALMT1 gene in cultured tobacco cells did improve Al tolerance. To test whether Al tolerance might be provided by the wheat ALMT1 gene in whole tobacco plants, the coding region of the wheat ALMT1 gene was excised from pTH-ALMT1-1 by digestion with NotI and SalI and blunted by end-filling. This fragment was then introduced into the SmaI site of pART7 and a plasmid that contained the ALMT1 gene in the correct orientation with respect to the 35S promoter and ocs terminator was identified. A NotI fragment containing the promoter:ALMT1:ocs expression cassette was introduced into the NotI site of the binary vector pPLEX502.

The binary vector was introduced into Agrobacterium by triparental mating and used to transform tobacco. Transgenic tobacco plants were selected on medium that contained kanamycin (100×g/mL) and clonal populations of the three highest expressing primary transformants were generated to enable experiments to be undertaken on genetically identical plants. Plants were transferred to tissue culture pots that contained liquid MS medium and roots allowed to grow over two weeks. The MS solution was then removed and replaced with a solution consisting of 50 μM AlCl₃ in 0.2 mM CaCl₂ pH 4.3. At intervals of 2 h, the solutions were replaced and the accumulated malate assayed using previously described procedures (Delhaize et al., 2004; Ryan et al., 1995).

FIG. 20 shows the malate efflux at each time interval for a representative transgenic line expressing ALMT1 (Line 1) and a control line transformed with the vector pPLEX502 lacking an insert. Similar data were obtained when root apices were assayed in isolation. Although some malate efflux was observed up to 4 hr, the level was relatively low and declined by 6 hr and afterward. It was considered that the relatively low levels of malate efflux combined with the rapid decline over time (relative to the growth period for the plants) were unlikely to provide effective Al tolerance to the tobacco plants. It therefore appeared that the ALMT1 gene did not provide tolerance to plants such as rice and tobacco but, in contrast, did provide tolerance to barley.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Anoop et al. (2003). Plant Physiol. 132, 2205-2217.
Chiu et al. (1996). Cur. Biol. 6, 325-330.
Culvenor. et al. (2004). Aust. J. Agric. Res (In Press),
de la Fuente et al. (1997). Science 276, 1566-1568.
Delhaize et al. (2000). Plant Physiol. 125, 2059-2067.
Delhaize et al. (2001). Ann. Rev. Plant Physiol. Plant Mol. Biol. 52, 527-560.
Delhaize et al. (2004). Proc. Natl. Acad. Sci. USA. 101, 15249-15254.
Echart et al. (2002). Euphytica 126, 309-313.
Edwards et al. (1991). Nucl. Acids Res. 19, 1349.
Katsoka et al. (2001). Plant Cell Environ. 25, 453-460.
Kochian (1995). Annu. Rev, Plant Physiol. Plant Mol. Bio. 46, 237-260.
Koyama et al. (2000). Plant Cell Physiol. 41, 1030-1037.
Needleman and Wunsch (1970). J. Mol. Biol. 48, 443-453.
Polle et al. (1978). Crop Sci. 18, 823-827.
Ryan et al. (1993). J. Exp. Bot. 44, 437-446.
Ryan et al. (1995a). Planta 196, 103-110.
Ryan et al. (1995b). Aust. J. Plant Physiol. 22, 531-536.
Ryan et al. (2001). Annu. Rev. Plant Physiol. Plant Mol. Biol. 52, 527-560.
Sasaki et al. (2004). Plant J. 37, 645-653.
Schenk et al. (2000). Proc. Natl. Acad. Sci. 97, 11655-116600.
Schunmann et al. (2003). Funct. Plant Biol. 30, 453-460.
Tesfaye et al. (2001). Plant Physiol. 127, 1836-1844.
Tingay et al. (1997). Plant J. 11, 1369-1376.
von Uexküll and Mutert (1995). Plant Soil 171, 1-15.
Wan and Lemaux (1994) Plant Physiol. 104, 37-48.
Wang et al. (1998). Acta Hort. 461, 401-405.
Zhao et al. (2003). Planta 217, 794-800.

1-41. (canceled)

42. A process for producing barley grain comprising:
a) growing a barley plant which is i) transgenic for an exogenous nucleic acid molecule that encodes a polypeptide whose amino acid sequence is set forth in SEQ ID NO:1, or a polypeptide having at least 95% identity to the amino acid sequence as set forth in SEQ ID NO:1, ii) which has enhanced tolerance to aluminium when the exogenous nucleic acid molecule is expressed relative to an isogenic barley plant not having the exogenous nucleic molecule, and iii) which produces barley grain that grows into barley plants whose roots grow at least 50% as long in a medium consisting of a hydroponic growth solution having at least 5 μM of aluminium chloride as the length of roots of an isogenic barley plant not having the exogenous nucleic acid when grown in a medium consisting of said hydroponic growth solution without aluminium chloride, wherein said hydroponic growth solution consists of distilled or deionized water with added salts at the concentrations: 500 μM KNO₃, 500 μM CaCl₂, 500 μM NH₄NO₃, 150 μM MgSO₄, 10 μM KH₂PO₄, 2 μM FeCl₃, 11 μM H₃BO₃, 2 μM MnCl₂, 0.35 μM ZnCl₂ and 0.2 μM CuCl₂ and which is adjusted to pH 4.3, and
b) harvesting the transgenic barley grain from the barley plant.

43. The process of claim 42, wherein the barley plant is homozygous for the exogenous nucleic acid molecule.

44. The process of claim 42, wherein the barley plant is grown in an acidic soil.

45. The process of claim 44, wherein the barley plant is grown in soil having at least 1 μM solubilised trivalent cation Al³⁺.

46. Barley grain produced according to the process of claim 42, the barley grain comprising the exogenous nucleic acid molecule.

47. A process for producing barley flour, wholemeal, starch or malt, the process comprising:
a) obtaining barley grain according to claim 46, and
b) extracting flour, wholemeal, or starch from the barley grain, or malting the barley grain.

48. Barley flour or wholemeal produced from the barley grain of claim 46, the flour or wholemeal comprising the exogenous nucleic acid molecule.

48. The process of claim 42, wherein the exogenous nucleic acid molecule encodes a polypeptide whose amino acid sequence is set forth in SEQ ID NO:1.

49. The process of claim 48, wherein the barley plant is homozygous for the exogenous nucleic acid molecule.

50. The process of claim 48, wherein the barley plant is grown in an acidic soil.

51. The process of claim 50, wherein the barley plant is grown in soil having at least 1 μM solubilised trivalent cation Al³⁺.

52. Barley grain produced according to the process of claim 48, the barley grain comprising the exogenous nucleic acid molecule.

53. A process for producing barley flour, wholemeal, starch or malt, the process comprising:
a) obtaining barley grain according to claim 52, and
b) extracting flour, wholemeal, or starch from the barley grain, or malting the barley grain.

54. Barley flour or wholemeal produced from the barley grain of claim 52, the flour or wholemeal comprising the exogenous nucleic acid molecule.
